(19) United States
(12) Patent Application Publication
(10) Pub. No.: US 2006/0160187 A1
(43) Pub. Date: Jul. 20, 2006

(54) Combinatorial interleukin-2 muteins

(75) Inventors: Kimberly Denis-Mize, Concord, CA (US); Susan E. Wilson, Alameda, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 11/301,276

(22) Filed: Dec. 12, 2005

Related U.S. Application Data
(63) Continuation of application No. 11/074,330, filed on Mar. 3, 2005, now abandoned.
(60) Provisional application No. 60/550,868, filed on Mar. 5, 2004.
(60) Provisional application No. 60/585,980, filed on Jul. 7, 2004.
(60) Provisional application No. 60/646,095, filed on Jan. 21, 2005.

(51) Int. Cl. C07K 14/54; C07H 21/04; C12P 21/04

(52) U.S. Cl. 435/69.52; 435/320.1; 435/325; 530/351; 536/23.5

(57) ABSTRACT

Novel human interleukin-2 (IL-2) muteins or variants thereof, and nucleic acid molecules and variants thereof are provided. Methods for producing these muteins as well as methods for stimulating the immune system of an animal are also disclosed. In addition, the invention provides recombinant expression vectors comprising the nucleic acid molecules of this invention and host cells into which expression vectors have been introduced. Pharmaceutical compositions are included comprising a therapeutically effective amount of a human IL-2 mutein of the invention and a pharmaceutically acceptable carrier. The IL-2 muteins have lower toxicity than native IL-2 or Proleukin® IL-2, while maintaining or enhancing NK cell-mediated effects, and can be used in pharmaceutical compositions for use in treatment of cancer, and in stimulating the immune response.

This application is a continuation application of U.S. patent application Ser. No. 11/074,330, filed Mar. 3, 2005 from which priority is claimed pursuant to 35 U.S.C. §120, and claims benefit under 35 U.S.C. §119(e) of provisional application 60/585,980 filed on Jul. 7, 2004, provisional application 60/550,868 filed on Mar. 5, 2004, and provisional application 60/646,095 filed on Jan. 21, 2005, which applications are hereby incorporated by reference in their entireties.

### FIELD OF THE INVENTION

The invention relates to muteins of human interleukin-2 (IL-2) having improved therapeutic efficacy. Also provided are methods for producing the novel molecules and pharmaceutical formulations that can be utilized to treat cancer and to stimulate the immune system of a mammal.

### BACKGROUND OF THE INVENTION

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al. (1987) N. Engl. J. Med. 316:889-897; Rosenberg (1988) Ann. Surg. 208:121-135; Topalian et al. (1988) J. Clin. Oncol. 6:839-853; Rosenberg et al. (1988) N. Engl. J. Med. 319:1676-1680; and Weber et al. (1992) J. Clin. Oncol. 10:33-40). However, high doses of IL-2 used to achieve positive therapeutic results with respect to tumor growth frequently cause severe side effects, including fever and chills, hypotension and capillary leak (vascular leak syndrome or VLS), and neurological changes (see, for example, Duggan et al. (1992) J. Immunotherapy 12:115-122; Gisselbrecht et al. (1994) Blood 83:2081-2085; and Sznol and Parkinson (1994) Blood 83:2020-2022).

Although the precise mechanism underlying IL-2-induced toxicity and VLS is unclear, accumulating data suggests that IL-2-induced natural killer (NK) cells trigger dose-limiting toxicities (DLT) as a consequence of overproduction of pro-inflammatory cytokines including IFN-α, IFN-γ, TNF-α, TNF-β, IL-1β, and IL-6. These cytokines activate monocytes/macrophages and induce nitric oxide production leading to subsequent damage of endothelial cells (Dubinett et al. (1994) Cell Immunol. 157:170-180; Samlowski et al. (1995) J. Immunother. Emphasis Tumor Immunol. 18:166-178). These observations have led others to develop IL-2 muteins that demonstrate preferential selectivity for T cells as opposed to NK cells based on the hypothesis that the high affinity IL-2 receptor (IL-2R) is selectively expressed on T cells (see, for example, BAY50-4798, the N88R IL-2 mutein of mature human IL-2 disclosed in International Publication No. WO 99/60128, and Shanafelt et al. (2000) Nat. Biotechnol. 18:1197-202).

Diverse NK effector functions such as natural (NK), LAK, and antibody-dependent (ADCC) cytolytic killing, cytokine production, and proliferation depend on the activation of specific intermediates in distinct NK intracellular signaling pathways. Importantly, evidence exists that selective modulation of IL-2-IL-2R interactions can influence diverse downstream NK- and T-cell-mediated effector functions such as proliferation, cytokine production, and cytolytic killing (Sauve et al. (1991) Proc. Natl. Acad. Sci. U.S.A. 88:4636-4640; Heaton et al. (1993) Cancer Res. 53:2597-2602; Eckenberg et al. (2000) J. Exp. Med. 191:529-540).

Proleukin® IL-2 (comprising the recombinant human IL-2 mutein aldesleukin; Chiron Corporation, Emeryville, Calif.) has been approved by the FDA to treat metastatic melanoma and renal carcinoma, and is being studied for other clinical indications, including non-Hodgkin's lymphoma, HIV, and breast cancer. However, due to the toxic side effects associated with IL-2, there is a need for a less toxic IL-2 mutein that allows greater therapeutic use of this interleukin. IL-2 muteins that have improved tolerability and/or enhanced IL-2-mediated NK cell or T cell effector functions would have broader use and would be particularly advantageous for cancer therapy and for modulating the immune response.

### BRIEF SUMMARY OF THE INVENTION

The invention relates to muteins of interleukin-2 (IL-2) that have improved functional profiles predictive of reduced toxicities. Isolated nucleic acid molecules encoding muteins of human IL-2 and isolated polypeptides comprising these muteins are provided. The muteins induce a lower level of pro-inflammatory cytokine production by NK cells while maintaining or increasing NK cell proliferation, maintaining NK-cell-mediated NK, LAK, and ADCC cytolytic functions, and maintaining T cell proliferative function as compared to the des-alanyl-1, C125S human IL-2 or C125S human IL-2 muteins. Clinical uses of these improved human IL-2 muteins in treatment of cancer and in modulating the immune response are also described.

In one aspect, the invention provides an isolated nucleic acid molecule comprising a nucleotide sequence encoding a mutein of human IL-2. In certain embodiments, the nucleic acid molecule encodes a mutein of human IL-2 comprising an amino acid sequence selected from the group consisting of SEQ ID NOS:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the invention includes an isolated nucleic acid molecule encoding a mutein of human IL-2 comprising a nucleotide sequence selected from the group consisting of the nucleotide sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71.

In certain embodiments, the invention includes an isolated nucleic acid molecule comprising a nucleotide sequence encoding a mutein of human IL-2, wherein the mutein has an amino acid sequence comprising residues 2-133 of a sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the invention includes an isolated nucleic acid molecule comprising a nucleotide sequence comprising nucleotides 4-399 of a sequence selected from the group consisting of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71.

In certain embodiments, the nucleic acid molecules described herein may further comprise a substitution, wherein nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71 are replaced with a triplet codon that encodes alanine.

In certain embodiments, the nucleic acid molecules described herein may further comprise a substitution, wherein nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71 are replaced with a triplet codon that encodes cysteine.

In certain embodiments, the nucleic acid molecules described herein are further modified to optimize expression. Such nucleic acids comprise a nucleotide sequence, wherein one or more codons encoding the mutein have been optimized for expression in a host cell of interest. Exemplary nucleic acids containing optimized codons may include, but are not limited to, a nucleic acid comprising a nucleotide sequence selected from the group consisting of SEQ ID NO:73, nucleotides 4-399 of SEQ ID NO:73, SEQ ID NO:74, and nucleotides 4-399 of SEQ ID NO:74.

The present invention further includes an expression vector for use in selected host cells, wherein the expression vector comprises one or more of the nucleic acids of the present invention. In such expression vectors, the nucleic acid sequences are operably linked to control elements compatible with expression in the selected host cell. Numerous expression control elements are known to those in the art, including, but not limited to, the following: transcription promoters, transcription enhancer elements, transcription termination signals, polyadenylation sequences, sequences for optimization of initiation of translation, and translation termination sequences. Exemplary transcription promoters include, but are not limited to those derived from polyoma, Adenovirus 2, cytomegalovirus, and Simian Virus 40.

In another aspect, the invention provides cells comprising the expression vectors of the present invention, wherein the nucleic acid sequence (e.g., encoding a mutein of human IL-2) is operably linked to control elements compatible with expression in the selected cell. In one embodiment, such cells are mammalian cells. Exemplary mammalian cells include, but are not limited to, Chinese hamster ovary cells (CHO) or COS cells. Other cells, cell types, tissue types, etc., that may be useful in the practice of the present invention include, but are not limited to, those obtained from the following: insects (e.g., Trichoplusia ni (Tn5) and Sf9), bacteria, yeast, plants, antigen presenting cells (e.g., macrophage, monocytes, dendritic cells, B-cells, T-cells, stem cells, and progenitor cells thereof), primary cells, immortalized cells, tumor-derived cells.

In another aspect, the present invention provides compositions comprising any of the expression vectors and host cells of the present invention for recombinant production of the human IL-2 muteins. Such compositions may include a pharmaceutically acceptable carrier.

In a further aspect, the invention provides an isolated polypeptide comprising a mutein of human IL-2. In certain embodiments, the invention includes an isolated polypeptide comprising an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the invention includes an isolated polypeptide comprising amino acid residues 2-133 of an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the polypeptides described herein may further comprise a substitution, wherein an alanine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In certain embodiments, the polypeptides described herein may further comprise a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In certain embodiments, the invention includes an isolated polypeptide comprising a mutein of human IL-2, wherein the mutein comprises the amino acid sequence set forth in SEQ ID NO:4 with a serine substituted for cysteine at position 125 of SEQ ID NO:4 and at least two additional amino acid substitutions within SEQ ID NO:4, wherein the mutein: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2. Exemplary combination substitutions include, but are not limited to, 19D40D, 19D81K, 36D42R, 36D61R, 36D65L, 40D36D, 40D61R, 40D65Y, 40D72N, 40D80K, 40G36D, 40G65Y, 80K36D, 80K65Y, 81K36D, 81K42E 81K61R, 81K65Y, 81K72N, 81K88D, 81K91D, 81K107H, 81L107H, 91N95G, 107H36D, 107H42E, 107H65Y, 107R36D, 107R72N, 40D81K107H, 40G81K107H, and 91N94Y95G. In certain embodiments, the mutein further comprises a deletion of alanine at position 1 of SEQ ID NO:4.

Increased proliferation of natural killer (NK) cells and decreased levels of pro-inflammatory cytokine production by NK cells can be detected using a NK-92 bioassay. The effects of the polypeptides described herein on proliferation of NK cells and pro-inflammatory cytokine production by NK cells are compared with the effects of a similar amount of des-alanyl-1, C125S human IL-2 or C125S human IL-2 under comparable assay conditions. In certain embodiments, an NK-92 bioassay is used to show that the polypeptides described herein induce a decreased level of the pro-inflammatory cytokine TNF-α relative to that observed for a similar amount of des-alanyl-1, C125S human IL-2 or C125S human IL-2 under comparable assay conditions.

In certain embodiments, a NK3.3 cytotoxicity bioassay is used to show that the polypeptides described herein maintain or improve human NK cell-mediated natural killer cytotoxicity, lymphokine activated killer (LAK) cytotoxicity, or ADCC-mediated cytotoxicity relative to that observed for a similar amount of des-alanyl-1, C125S human IL-2 mutein or C125S human IL-2 under comparable assay conditions.

In certain embodiments, the NK cell proliferation induced by the mutein is greater than 150% of that induced by a similar amount of des-alanyl-1, C125S human IL-2 or C125S human IL-2 under comparable assay conditions.

In certain embodiments, the NK cell proliferation induced by the mutein is greater than 170% of that induced by des-alanyl-1, C125S human IL-2 or C125S human IL-2.

In certain embodiments, the NK cell proliferation induced by the mutein is about 200% to about 250% of that induced by des-alanyl-1, C125S human IL-2 or C125S human IL-2.

In certain embodiments, the NK cell proliferation induced by the mutein is increased by at least 10% over that induced by a similar amount of des-alanyl-1, C125S human IL-2 or C125S human IL-2 under comparable assay conditions.

In certain embodiments, the NK cell proliferation induced by the mutein is increased by at least 15% over that induced by des-alanyl-1, C125S human IL-2 or C125S human IL-2.

In certain embodiments, the pro-inflammatory cytokine production induced by the mutein is less than 100% of that induced by a similar amount of des-alanyl-1, C 125S human IL-2 or C125S human IL-2 under similar assay conditions.

In certain embodiments, the pro-inflammatory cytokine production induced by the mutein is less than 70% of that induced by des-alanyl-1, C125S human IL-2 or C125S human IL-2.

In certain embodiments, the invention includes an isolated polypeptide comprising a mutein of human IL-2, wherein the mutein comprises the amino acid sequence set forth in SEQ ID NO:4 with a serine substituted for cysteine at position 125 of SEQ ID NO:4 and at least two additional amino acid substitutions within SEQ ID NO:4, wherein the ratio of IL-2-induced NK cell proliferation to IL-2-induced TNF-α production of the mutein is at least 1.5-fold greater than that observed for a similar amount of des-alanyl-1, C125S human IL-2 mutein or C125S human IL-2 mutein under comparable assay conditions, wherein NK cell proliferation at 0.1 nM mutein and TNF-α production at 1.0 nM mutein are assayed using the NK-92 bioassay. In certain embodiments, the ratio is at least 2.5-fold greater than that observed for des-alanyl-1, C125S human IL-2 or C125S human IL-2. In certain embodiments, the ratio is at least 3.0-fold greater than that observed for des-alanyl-1, C125S human IL-2 or C125S human IL-2.

In certain embodiments, the invention includes an isolated polypeptide comprising an amino acid sequence for a mutein of human IL-2, wherein the mutein comprises the amino acid sequence set forth in SEQ ID NO:4 with a serine substituted for cysteine at position 125 of SEQ ID NO:4 and with at least two additional amino acid substitutions, wherein the additional substitutions reside at positions of SEQ ID NO:4 selected from the group consisting of positions 19, 36, 40, 42, 61, 65, 72, 80, 81, 88, 91, 95, and 107. Exemplary combination substitutions include, but are not limited to, 19D40D, 19D81K, 36D42R, 36D61R, 36D65L, 40D36D, 40D61R, 40D65Y, 40D72N, 40D80K, 40G36D, 40G65Y, 80K36D, 80K65Y, 81K36D, 81K42E 81K61R, 81K65Y, 81K72N, 81K88D, 81K91D, 81K107H, 81L107H, 91N95G, 107H36D, 107H42E, 107H65Y, 107R36D, 107R72N, 40D81K107H, 40G81K107H, and 91N94Y95G. In certain embodiments, the polypeptide further comprises a deletion of alanine at position 1 of SEQ ID NO:4.

In another aspect, the invention provides a method of producing a mutein of human interleukin-2 (IL-2) comprising transforming a host cell with an expression vector comprising any of the nucleic acid molecules described herein; culturing the host cell in a cell culture medium under conditions that allow expression of the nucleic acid molecule as a polypeptide; and isolating the polypeptide. In certain embodiments, the mutein of human interleukin-2 (IL-2) is capable of maintaining or enhancing proliferation of NK cells and also induces a lower level of pro-inflammatory cytokine production by NK cells as compared with a similar amount of a reference IL-2 mutein selected from des-alanyl-1, C125S human IL-2 and C125 human IL-2 under similar assay conditions, wherein the NK cell proliferation and pro-inflammatory cytokine production are assayed using the NK-92 bioassay.

In another aspect, the invention provides compositions comprising a therapeutically effective amount of one or more of the polypeptides described herein comprising a mutein of human IL-2. Such compositions may further include a pharmaceutically acceptable carrier.

In another aspect, the invention provides a method for stimulating the immune system of a mammal, comprising administering to the mammal a therapeutically effective amount of a human IL-2 mutein, wherein said mutein induces a lower level of pro-inflammatory cytokine production by NK cells and maintains or enhances NK cell proliferation compared to a similar amount of a reference IL-2 mutein selected from des-alanyl-1, C125S human IL-2 and C125S human IL-2 under comparable assay conditions, wherein said NK cell proliferation and said pro-inflammatory cytokine production are assayed using the NK-92 bioassay. In certain embodiments, the mammal is a human.

In certain embodiments, the human IL-2 mutein used to stimulate the immune system comprises an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the human IL-2 mutein used to stimulate the immune system comprises an amino acid sequence comprising residues 2-133 of an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the human IL-2 mutein used to stimulate the immune system may further comprise a substitution, wherein an alanine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In certain embodiments, the human IL-2 mutein used to stimulate the immune system may further comprise a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In another aspect, the invention provides a method for treating a cancer in a mammal, comprising administering to the mammal a therapeutically effective amount of a human IL-2 mutein, wherein the mutein induces a lower level of pro-inflammatory cytokine production by NK cells and maintains or enhances NK cell proliferation compared to a similar amount of a reference IL-2 mutein selected from des-alanyl-1, C125S human IL-2 and C125S human IL-2 under similar assay conditions, wherein the NK cell proliferation and said pro-inflammatory cytokine production are assayed using the NK-92 bioassay. In certain embodiments, the mammal is a human.

In certain embodiments, the human IL-2 mutein used for treating a cancer may comprise an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the human IL-2 mutein used for treating a cancer may comprise an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In certain embodiments, the human IL-2 mutein used for treating a cancer may further comprise a substitution, wherein an alanine residue is substituted for the serine residue at position 125 of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In certain embodiments, the human IL-2 mutein used for treating a cancer may further comprise a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In another aspect, the invention provides a method for reducing interleukin-2 (IL-2)-induced toxicity symptoms in a subject undergoing IL-2 administration as a treatment protocol. The method of treatment comprises administering IL-2 as an IL-2 mutein.

In certain embodiments, the IL-2 mutein used in treatment comprises an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72.

In certain embodiments, the IL-2 mutein used in treatment comprises residues 2-133 of an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;

In certain embodiments, the IL-2 mutein used in treatment further comprises a substitution, wherein an alanine residue is substituted for the serine residue at position 125 of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

In certain embodiments, the IL-2 mutein used in treatment further comprises a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

### BRIEF DESCRIPTION OF THE DRAWINGS

FIG. 1 outlines the schematic for compilation of the combination proliferation/pro-inflammatory cytokine production assay procedure used with IL-2 mutein-stimulated human PBMC isolated from normal human donors.

FIG. 2 shows proliferation and TNF-α production mediated by 40D72N IL-2 mutein in human PBMC.

FIG. 3 shows proliferation and TNF-α production mediated by 40D61R IL-2 mutein in human PBMC.

FIG. 4 shows maintenance of human NK-mediated LAK and ADCC activity for IL-2 mutein-stimulated human PBMC isolated from normal human donors.

### DETAILED DESCRIPTION OF THE INVENTION

The present invention is directed to novel muteins of human interleukin-2 (IL-2) that have improved therapeutic efficacy due to their reduced toxicity and/or improved NK or T cell effector functions. The human IL-2 muteins disclosed herein, and biologically active variants thereof, elicit reduced pro-inflammatory cytokine production while maintaining or increasing natural killer (NK) cell proliferation, as compared to the desalanyl-1, C125S human IL-2 mutein or the C125S human IL-2 mutein. By “pro-inflammatory cytokine” is intended a cytokine that is able to stimulate the immune system. Such cytokines include, but are not limited to, IFN-α, IFN-γ, TNF-α, TNF-β, IL-1β, and IL-6.

The term “mutein” refers to a protein comprising a mutant amino acid sequence that differs from the amino acid sequence for the naturally occurring protein by amino acid deletions, substitutions, or both. The human IL-2 muteins of the present invention comprise an amino acid sequence that differs from the mature human IL-2 sequence by having a serine residue substituted for the cysteine residue at position 125 of the mature human IL-2 sequence (i.e., C125S) and at least two additional amino acid substitutions, and may further comprise one or more amino acid deletions relative to the mature human IL-2 sequence, such as deletion of the N-terminal alanine (Ala) at position 1 of the mature human IL-2 protein. In alternative embodiments, the human IL-2 muteins of the present invention retain the cysteine residue at position 125 of the mature human IL-2 sequence but have at least two other amino acid substitutions, and may further comprise one or more amino acid deletions relative to the mature human IL-2 sequence, such as deletion of the N-terminal alanine (Ala) at position 1 of the mature human IL-2 protein. These human IL-2 muteins can be glycosylated or unglycosylated depending upon the host expression system used in their production. The particular substitutions disclosed herein result in a human IL-2 variant that retains the desired activities of eliciting reduced pro-inflammatory cytokine production while maintaining or increasing NK cell proliferation, as compared to the des-alanyl-1, C125S human IL-2 mutein or the C125S human IL-2 mutein using the NK-92 cell assays described herein. Having identified the positions within the human IL-2 sequence and the relevant substitutions at these positions that result in an IL-2 variant with reduced toxicity and/or improved NK cell proliferation, it is within the skill of one in the art to vary other residues within the human IL-2 sequence to obtain variants of the human IL-2 muteins disclosed herein that also retain these desired activities. Such variants of the human IL-2 muteins disclosed herein are also intended to be encompassed by the present invention, and are further defined below.

Human IL-2 is initially translated as a precursor polypeptide, shown in SEQ ID NO:2, which is encoded by a nucleotide sequence such as that set forth in SEQ ID NO:1. The precursor polypeptide includes a signal sequence at residues 1-20 of SEQ ID NO:2. The term “mature human IL-2” refers to the amino acid sequence set forth as SEQ ID NO:4, which is encoded by a nucleotide sequence such as that set forth as SEQ ID NO:3. The terms “C125S human IL-2 mutein” or “C125S human IL-2” refer to a mutein of mature human IL-2 that retains the N-terminal alanine residing at position 1 of the mature human IL-2 sequence and which has a substitution of serine for cysteine at position 125 of the mature human IL-2 sequence. C125S human IL-2 mutein has the amino acid sequence set forth in SEQ ID NO:6, which is encoded by a nucleotide sequence such as that set forth as SEQ ID NO:5. The terms “des-alanyl-1, C125S human IL-2” and “des-alanyl-1, serine-125 human IL-2” refer to a mutein of mature human IL-2 that has a substitution of serine for cysteine at amino acid position 125 of the mature human IL-2 sequence and which lacks the N-terminal alanine that resides at position 1 of the mature human IL-2 sequence (i.e., at position 1 of SEQ ID NO:4). Des-alanyl-1, C125S human IL-2 has the amino acid sequence set forth in SEQ ID NO:8, which is encoded by a nucleotide sequence such as that set forth in SEQ ID NO:7. The E. coli recombinantly produced des-alanyl-1, C125S human IL-2 mutein, which is referred to as “aldesleukin,” is available commercially as a formulation that is marketed under the tradename Proleukin® IL-2 (Chiron Corporation, Emeryville, Calif.). For the purposes of the present invention, the des-alanyl-1, C125S human IL-2 and C125S human IL-2 muteins serve as reference IL-2 muteins for determining the desirable activities that are to be exhibited by the human IL-2 muteins of the invention. That is, the desired activity of reduced IL-2-induced pro-inflammatory cytokine production, particularly TNF-α production, by NK cells in a suitable human IL-2 mutein of the invention is measured relative to the amount of pro-inflammatory cytokine production of NK cells that is induced by an equivalent amount of the des-alanyl-1, C125S human IL-2 mutein or C125S human IL-2 mutein under similar assay conditions. Similarly, the desired activity of maintaining or increasing IL-2-induced NK cell proliferation in a suitable human IL-2 mutein of the invention is measured relative to the amount of NK cell proliferation induced by an equivalent amount of the des-alanyl-1, C125S human IL-2 mutein or C125S human IL-2 mutein under similar assay conditions.

Isolated nucleic acid molecules encoding human IL-2 muteins, and biologically active variants thereof, comprising the amino acid sequence of des-alanyl-1, C125S human IL-2 (SEQ ID NO:8) or C125S human IL-2 (SEQ ID NO:6) with at least two additional amino acid substitutions, and which induce a lower level of pro-inflammatory cytokine production by NK cells while maintaining or increasing NK cell proliferation, as compared to these two reference IL-2 muteins, are provided. The isolated polypeptides encoded by the nucleic acid molecules of the invention are also provided.

Human IL-2 muteins of the invention include the muteins set forth in SEQ ID NOS:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72, which are also referred to herein as “the sequences set forth in even SEQ ID NOS:10-72.” The present invention also provides any nucleotide sequences encoding these muteins, for example, the coding sequences set forth in SEQ ID NOS:9, 11, 13, 15, 17, 19, 21, 23, 25, 27, 29, 31, 33, 35, 37,39, 41, 43, 45, 47, 49, 51, 53, 55, 57, 59, 61, 63, 65, 67, 69, and 71, respectively. These coding sequences are also referred to herein as “the sequences set forth in odd SEQ ID NOS:9-71.” The muteins set forth in these foregoing amino acid sequences comprise the C125S human IL-2 amino acid sequence with at least two additional substitutions, where these additional substitutions are represented by the combination substitutions shown in Table 1 below. By “combination substitution” is intended a group of two or more residue substitutions that occur within the human IL-2 mutein sequence. Thus, for example, the combination substitution designated as “19D40D” is intended to mean the human IL-2 mutein of the invention comprises both a substitution of an aspartic acid residue (i.e., D) for the leucine residue at the position corresponding to position 19 of mature human IL-2 (shown in SEQ ID NO:4) and a substitution of an aspartic acid residue (i.e., D) for the leucine residue at the position corresponding to position 40 of mature human IL-2 (shown in SEQ ID NO:4). Similarly, the combination substitution designated as “40D81K107H” is intended to mean the human IL-2 mutein of the invention comprises all three of the following substitutions: a substitution of an aspartic acid residue (i.e., D) for the leucine residue at the position corresponding to position 40 of mature human IL-2, a substitution of a lysine residue (i.e., K) for the arginine residue at the position corresponding to position 81 of mature human IL-2, and a substitution of a histidine residue (i.e., H) for the tyrosine residue at the position corresponding to position 107 of mature human IL-2. In alternative embodiments, the human IL-2 muteins of the present invention have the initial alanine residue at position 1 of these amino acid sequences deleted, and thus comprise the des-alanyl-1, C125S human IL-2 amino acid sequence with at least two additional substitutions, where these additional substitutions are represented by the combination substitutions shown in Table 1 below. These muteins thus have an amino acid sequence that comprises residues 2-133 of the sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72. The present invention also provides any nucleotide sequences encoding these muteins, for example, the coding sequences set forth in nucleotides 4-399 of the sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71.

Biologically active variants of the human IL-2 muteins of the invention, including fragments and truncated forms thereof, that have the desired human IL-2 mutein functional profile as noted herein are also provided. For example, fragments or truncated forms of the disclosed human IL-2 muteins may be generated by removing amino acid residues from the full-length human IL-2 mutein amino acid sequence using recombinant DNA techniques well known in the art and described elsewhere herein. Suitable variants of the human IL-2 muteins of the invention will have biological activities similar to those exhibited by the novel human IL-2 muteins themselves, i.e., they have a low toxicity of the novel human IL-2 mutein (i.e., low or reduced pro-inflammatory cytokine production), as well as the ability to maintain or increase NK cell proliferation, when compared to the reference IL-2 molecule, i.e., des-alanyl-1, C125S or C125S human IL-2, using the bioassays disclosed elsewhere herein. It is recognized that a variant of any given novel human IL-2 mutein identified herein may have a different absolute level of a particular biological activity relative to that observed for the novel human IL-2 mutein of the invention, so long as it retains the desired biological profile of having reduced toxicity, that is, it induces a lower level of pro-inflammatory cytokine production by NK cells, and/or increased NK cell proliferation when compared to the reference human IL-2 mutein.

TABLE 1
Examples of human IL-2 muteins of the invention that comprise
the amino acid sequence of C125S human IL-2 (SEQ ID NO: 6)
or des-alanyl-1, C125S human IL-2 (SEQ ID NO: 8) with at least
two additional substitutions, where the additional substitutions
are selected from the combination substitutions shown below.
Residue position is relative to the position within the mature
human IL-2 sequence set forth in SEQ ID NO: 4. These residue
positions also correspond to the position within the C125S
human IL-2 sequence set forth in SEQ ID NO: 6. Each residue
position is followed by the first letter abbreviation for the
amino acid that has been substituted for the naturally occurring
residue at that position.

| | |
|---|---|
| 19D40D | 81K61R |
| 19D81K | 81K65Y |
| 36D42R | 81K72N |
| 36D61R | 81K88D |
| 36D65L | 81K91D |
| 40D36D | 81K107H |
| 40D61R | 81L107H |
| 40D65Y | 91N95G |
| 40D72N | 107H36D |
| 40D80K | 107H42E |
| 40G36D | 107H65Y |
| 40G65Y | 107R36D |
| 80K36D | 107R72N |
| 80K65Y | 40D81K107H |
| 81K36D | 40G81K107H |
| 81K42E | 91N94Y95G |

Compositions of the invention further comprise vectors and host cells for the recombinant production of the human IL-2 muteins of the invention or biologically active variants thereof. In addition, pharmaceutical compositions comprising a therapeutically effective amount of a human IL-2 mutein disclosed herein or biologically active variant thereof, and a pharmaceutically acceptable carrier, are also provided.

Methods for producing muteins of human IL-2 that induce a lower level of pro-inflammatory production by NK cells and which maintain or increase NK cell proliferation relative to that observed for the reference IL-2 muteins are encompassed by the present invention. These methods comprise transforming a host cell with an expression vector comprising a nucleic acid molecule encoding a novel human IL-2 mutein of the invention, or encoding a biologically active variant thereof, culturing the host cell in a cell culture medium under conditions that allow expression of the encoded polypeptide, and isolating the polypeptide product.

Methods are also provided for stimulating the immune system of an animal, or for treating a cancer in a mammal, comprising administering to the animal a therapeutically effective amount of a human IL-2 mutein of the invention, or biologically active variant thereof, wherein the IL-2 mutein or variant thereof induces a lower level of pro-inflammatory cytokine production by NK cells, and maintains or increases NK cell proliferation compared to des-alanyl-1, C125S human IL-2 or C125S human IL-2 as determined using the bioassays disclosed herein below.

The present invention also provides a method for reducing interleukin-2 (IL-2)-induced toxicity symptoms in a subject undergoing IL-2 administration as a treatment protocol. The method comprises administering an IL-2 mutein of the present invention, i.e., a mutein that induces a lower level of pro-inflammatory cytokine production by NK cells, and which maintains or increases NK cell proliferation compared to des-alanyl-1, C125S human IL-2 or C125S human IL-2 as determined using the bioassays disclosed herein below.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein. When the protein of the invention or biologically active variant thereof is recombinantly produced, preferably culture medium represents less than about 30%, 20%, 10%, 5%, or 1% (by dry weight) of chemical precursors or non-protein-of-interest chemicals.

Biological Activity of Novel Human IL-2 Muteins

The novel human IL-2 muteins of the present invention have an increased therapeutic index compared to the des-alanyl-1, C125S human IL-2 mutein, or compared to the C125S human IL-2 mutein. The latter two muteins are referred to herein as “reference IL-2 muteins,” as the biological profiles of the novel muteins of the invention are compared to the biological profiles of these two previously characterized human IL-2 muteins, where any given comparison is made using similar protein concentrations and comparable assay conditions, in order to classify the muteins of the present invention. The increased therapeutic index of the muteins of the present invention is reflected in an improved toxicity profile (i.e., the mutein induces a lower level of pro-inflammatory cytokine production by NK cells), an increased NK and/or T cell effector function without increased toxicity, or both an improved toxicity profile and an increased NK and/or T cell effector function of these muteins when compared to the toxicity profile and NK and/or T cell effector function of either of these two reference IL-2 muteins.

Three functional endpoints were used to select the muteins with increased therapeutic index: (1) the ability to reduce IL-2-induced pro-inflammatory cytokine production by NK cells as compared to des-alanyl-1, C125S human IL-2 or C125S human IL-2; (2) the ability to maintain or increase IL-2-induced proliferation of NK and T cells without an increase in pro-inflammatory cytokine production by the NK cells as compared to des-alanyl-1, C125S human IL-2 or C125S human IL-2; and (3) the ability to maintain or improve (i.e., increase) NK-mediated cytolytic cell killing as compared to des-alanyl-1, C125S human IL-2 or C125S human IL-2. NK-mediated cytolytic cell killing includes NK-mediated, lymphokine activated killer (LAK)-mediated, and antibody-dependent cellular cytotoxicity (ADCC)-mediated cytolytic killing.

The novel human IL-2 muteins disclosed herein that exhibit the greatest improvements in therapeutic index fall within three functional classes predictive of improved clinical benefit. Of note is that all of these muteins exhibit maintained or increased T cell proliferation activity and NK-mediated cytolytic activity. The first functional class of muteins is characterized by having beneficial mutations that reduce IL-2-induced pro-inflammatory cytokine production by NK cells as compared to a reference IL-2 mutein, i.e., des-alanyl-1, C125S human IL-2 or C125S human IL-2, while maintaining IL-2-induced NK cell proliferation. The second functional class of muteins increases IL-2-induced NK cell proliferation relative to that induced by either of the reference IL-2 muteins, without negatively impacting (i.e., increasing) pro-inflammatory cytokine production relative to that induced by either of the reference IL-2 muteins. The third functional class of muteins includes muteins that are “bi-functional” in that they are able to reduce IL-2-induced pro-inflammatory cytokine production by NK cells while increasing IL-2-induced NK cell proliferation when compared to the levels of these activities induced by either of these two reference IL-2 muteins.

Assays to measure IL-2-induced NK cell proliferation and pro-inflammatory cytokine production by freshly isolated NK cells are well known in the art. See, for example, Perussia (1996) Methods 9:370 and Baume et al. (1992) Eur. J. Immunol. 22:1-6. The NK-92 cell line has phenotypic and functional characteristics of NK cells, including proliferation in the presence of IL-2 (Gong et al. (1994) Leukemia 8:652), however little or no production of TNF-α in the presence of IL-2 has previously been reported (Nagashima et al. (1998) Blood 91:3850). IL-2 bioassays that have been developed for screening functional activities of human NK and T cells are disclosed herein and in the Experimental section below. Though other assays can be used to measure NK cell proliferation and pro-inflammatory cytokine production of NK cells, and T cell effector function, preferably the IL-2 bioassays disclosed herein are used to screen IL-2 muteins of interest to determine whether they retain the desired characteristics of the muteins disclosed herein. Of particular interest is their decreased induction of TNF-α production by NK cells. Thus, in one embodiment, IL-2-induced NK cell proliferation and TNF-α production are measured using the IL-2 bioassay described herein below for the human NK-92 cell line (ATCC CRL-2407, CMCC ID #11925). For a description of the NK-92 cell line, see Gong et al. (1994) Leukemia 8(4):652-658. For purposes of the present invention, this bioassay is referred to as the “NK-92 bioassay.”

By “reduce” or “reduced” pro-inflammatory cytokine production is intended that the human IL-2 muteins of the invention induce a level of pro-inflammatory cytokine production by NK cells that is decreased relative to that induced by the reference IL-2 muteins, i.e., des-alanyl-1, C125S human IL-2 or C125S human IL-2 mutein, particularly with respect to induction of TNF-α production by NK cells. Though the human IL-2 muteins of the present invention induce a minimal level of TNF-α production by NK cells that is at least 20% of that induced by a similar amount of des-alanyl-1, C125S human IL-2 or C125S human IL-2 under comparable assay conditions, the maximal level of TNF-α production by NK cells that can be induced by a mutein of the present invention depends upon the functional class into which a mutein has been categorized.

Thus, for example, in some embodiments, the muteins have been selected for greatly enhanced induction of NK cell proliferation without having a negative impact on IL-2-induced TNF-α production by NK cells (i.e., the second functional class of muteins). In these embodiments, the human IL-2 muteins of the present invention induce a level of TNF-α production by NK cells that is similar to (i.e., ±10%) that induced by the reference IL-2 muteins or, preferably, less than 90% of that induced by the reference IL-2 muteins, where TNF-α production is assayed using the human NK-92 cell line (ATCC CRL-2407, CMCC ID #11925) (i.e., using the NK-92 bioassay disclosed herein) and a 1.0 nM or 100 pM (i.e., 0.1 nM) concentration of the respective human IL-2 muteins. In other embodiments of the invention, the human IL-2 muteins of the present invention induce a level of TNF-α production by NK cells that is less than 90%, preferably less than 85%, even more preferably less than 80% of the TNF-α production induced by a similar amount of des-alanyl-1, C125S human IL-2 or C125S human IL-2 under comparable assay conditions, where TNF-α production is assayed using the human NK-92 cell line (i.e., using the NK-92 bioassay disclosed herein) and a 1.0 nM concentration of the respective human IL-2 muteins. In some embodiments, the human IL-2 muteins of the invention induce at least 20% but less than 60% of the TNF-α production induced by des-alanyl-1, C125S human IL-2 or C125S human IL-2, where TNF-α production is assayed using the human NK-92 cell line (i.e., using the NK-92 bioassay disclosed herein) and a 1.0 nM concentration of the respective human IL-2 muteins. Such muteins, which also maintain or increase IL-2-induced NK cell proliferation relative to the reference IL-2 muteins, fall within the first functional class of IL-2 muteins.

By “maintain” is intended that the human IL-2 muteins of the present invention induce at least 70%, preferably at least 75%, more preferably at least 80%, and most preferably at least 85% and up to and including 100% (i.e., equivalent values) of the desired biological activity relative to the level of activity observed for a similar amount of des-alanyl-1, C125S human IL-2 or C125S human IL-2 under comparable assay conditions. Thus, where the desired biological activity is induction of NK cell proliferation, suitable IL-2 muteins of the invention induce a level of NK cell proliferation that is at least 70%, preferably at least 75%, more preferably at least 80%, and most preferably at least 85%, 90%, 95% and up to and including 100% (±5%) of the NK cell proliferation activity induced by a similar amount of des-alanyl-1, C125S human IL-2 or C125S human IL-2, where NK cell proliferation is assayed under comparable conditions using the same bioassay (i.e., the NK-92 bioassay disclosed herein) and similar amounts of these IL-2 muteins.

By “enhance” or “increase” or “improve” is intended that the human IL-2 mutein induces the desired biological activity at a level that is increased relative to that observed for a similar amount of des-alanyl-1, C125S human IL-2 or C125S human IL-2 under comparable assay conditions. Thus, where the desired biological activity is induction of NK cell proliferation, suitable IL-2 muteins of the invention induce a level of NK cell proliferation that is at least 105%, 110%, 115%, more preferably at least 120%, even more preferably at least 125%, and most preferably at least 130%, 140%, or 150% of the NK cell proliferation activity observed for a similar amount of des-alanyl-1, C125S human IL-2 or C125S human IL-2 using the same NK cell proliferation assay (for example, the NK-92 bioassay disclosed herein).

Assays to measure NK cell proliferation are well known in the art (see, for example, Baume et al. (1992) Eur. J. Immuno. 22:1-6, Gong et al. (1994) Leukemia 8(4):652-658, and the NK-92 bioassay described herein). Preferably NK-92 cells are used to measure IL-2-induced pro-inflammatory cytokine production, particularly TNF-α production, and NK cell proliferation (i.e., the NK-92 bioassay disclosed herein). Suitable concentrations of human IL-2 mutein for use in the NK cell proliferation assay include about 0.005 nM (5 pM) to about 1.0 nM (1000 pM), including 0.005 nM, 0.02 nM, 0.05 nM, 0.1 nM, 0.5 nM, 1.0 nM, and other such values between about 0.005 nM and about 1.0 nM. In preferred embodiments described herein below, the NK cell proliferation assay is carried out using NK-92 cells and a concentration of human IL-2 mutein of about 0.1 nM or about 1.0 nM.

As a result of their reduced induction of pro-inflammatory cytokine production and maintained or enhanced IL-2-induced NK cell proliferation, the human IL-2 muteins of the present invention have a more favorable ratio of IL-2-induced NK cell proliferation:IL-2-induced pro-inflammatory cytokine production by NK cells than does either des-alanyl-1, C125S human IL-2 or C125S human IL-2, where these activities are measured for each mutein using comparable protein concentrations and bioassay conditions. Where the pro-inflammatory cytokine being measured is TNF-α, suitable human IL-2 muteins of the invention have a ratio of IL-2-induced NK cell proliferation at 0.1 nM mutein:IL-2-induced TNF-α production by NK cells at 1.0 nM mutein that is at least 1.5-fold that obtained with des-alanyl-1, C125S human IL-2 or C125S human IL-2 under similar bioassay conditions and protein concentrations, more preferably at least 1.75-fold, 2.0-fold, 2.25-fold, even more preferably at least 2.75-fold, 3.0-fold, or 3.25-fold that obtained with the reference IL-2 muteins. In some embodiments, the human IL-2 muteins of the invention have a ratio of IL-2-induced NK cell proliferation at 0.1 nM mutein:IL-2-induced TNF-α production by NK cells at 1.0 nM mutein that is at least 3.5-fold, 3.75-fold, 4.0-fold, 4.5-fold, or even 5.0-fold that obtained with des-alanyl-1, human IL-2 mutein or C125S human IL-2 mutein under similar bioassay conditions and protein concentrations.

The muteins of the present invention may also enhance (i.e., increase) NK cell survival relative to that observed with des-alanyl-1, C125S human IL-2 or C125S human IL-2 under similar bioassay conditions and protein concentrations. NK cell survival can be determined using any known assay in the art, including the assays described herein. Thus, for example, NK cell survival in the presence of an IL-2 mutein of interest can be determined by measuring the ability of the IL-2 mutein to block glucocorticosteroid programmed cell death and induce BCL-2 expression in NK cells (see, for example, Armant et al. (1995) Immunology 85:331).

The present invention provides an assay for monitoring IL-2 effects on NK cell survival. Thus, in one embodiment, NK cell survival in the presence of a human IL-2 mutein of interest is determined by measuring the ability of the mutein to induce the cell survival signaling cascade in NK 3.3 cells (CMCC ID#12022; see Kombluth (1982) J. Immunol. 129(6):2831-2837) using a pAKT ELISA. In this manner, upregulation of AKT phosphorylation in NK cells by an IL-2 mutein of interest is used as an indicator of NK cell survival.

The IL-2 muteins for use in the methods of the present invention will activate and/or expand natural killer (NK) cells to mediate lymphokine activated killer (LAK) activity and antibody-dependent cellular cytotoxicity (ADCC). Resting (unactivated) NK cells mediate spontaneous or natural cytotoxicity against certain cell targets referred to as “NK-cell sensitive” targets, such as the human erythroleukemia K562 cell line. Following activation by IL-2, NK cells acquire LAK activity. Such LAK activity can be assayed, for example, by measuring the ability of IL-2-activated NK cells to kill a broad variety of tumor cells and other “LAK-sensitive/NK-insensitive” targets, such as the Daudi B-cell lymphoma line, that are normally resistant to lysis by resting (i.e., unactivated) NK cells. Similarly, ADCC activity can be assayed by measuring the ability of IL-2-activated NK cells to lyse “LAK-sensitive/NK-insensitive” target cells, such as Daudi B-cell lymphoma line, or other target cells not readily lysed by resting (i.e., unactivated) NK cells in the presence of optimal concentrations of relevant tumor cell specific antibodies. Methods for generating and measuring cytotoxic activity of NK/LAK cells and ADCC are known in the art. See for example, Current Protocols in Immunology: Immunologic Studies in Humans, Supplement 17, Unit 7.7, 7.18, and 7.27 (John Wiley & Sons, Inc., 1996), herein incorporated by reference. In one embodiment, the ADCC activity of the IL-2 muteins of the invention is measured using the NK3.3 cell line, which displays phenotypic and functional characteristics of peripheral blood NK cells. For purposes of the present invention, this assay is referred to herein as the “NK3.3 cytotoxicity bioassay.”

The human IL-2 muteins of the invention may also maintain or enhance IL-2-induced T cell proliferation compared to that observed for des-alanyl-1, C125S human IL-2 or C125S human IL-2 under similar bioassay conditions and protein concentrations. T cell proliferation assays are well known in the art. In one embodiment, the human T-cell line Kit225 (CMCC ID#11234; Hori et al (1987) Blood 70(4): 1069-1072) is used to measure T cell proliferation in accordance with the assay described herein below.

As noted above, the leading human IL-2 mutein candidates identified herein (i.e., those novel muteins having the most improved therapeutic index) fall within three functional classes. The first functional class includes those muteins that induce a lower level of TNF-α production by NK cells, about 60%, or less, of that induced by des-alanyl-1, C125S human IL-2 or C125S human IL-2 when all muteins are assayed under similar conditions at a protein concentration of 1.0 nM, and which maintain or enhance NK cell proliferation relative to des-alanyl-1, C125S human IL-2 or C125S human IL-2. These muteins can be further subdivided into two subclasses: (1) those human IL-2 muteins that enhance (i.e., greater than 100%) IL-2-induced NK cell proliferation relative to that observed for the reference human IL-2 muteins when these muteins are assayed under similar conditions at a protein concentration of about 1.0 nM, but which have reduced (i.e., less than 100%) NK cell proliferative activity relative to that observed for the reference human IL-2 muteins at concentrations of about 0.1 nM or below; and (2) those human IL-2 muteins that enhance (i.e., greater than 100%) or maintain (i.e., at least about 70% up to about 100%) the IL-2-induced NK cell proliferation relative to that observed for the reference human IL-2 muteins when these muteins are assayed under similar conditions at protein concentrations of about 1.0 nM down to about 0.05 nM (i.e., about 50 pM). In one embodiment, IL-2-induced NK proliferation and TNF-α production are determined using NK-92 cells (i.e., using the NK-92 bioassay disclosed herein), in which NK cell proliferation is determined using a commercially available MTT dye-reduction kit (CellTiter 96® Non-Radioactive Cell Proliferation Assay Kit; available from Promega Corp., Madison, Wis.) and a stimulation index is calculated based on a colorimetric readout; and TNF-α is quantified using a commercially available TNF-α ELISA kit (BioSource Cytoscreen™ Human TNF-α ELISA kit; Camarillo, Calif.). Human IL-2 muteins within subclass (1) of the first functional class include those muteins comprising the amino acid sequence of des-alanyl-1, C125S human IL-2 (SEQ ID NO:8) or C125S human IL-2 (SEQ ID NO:6) with at least one of the combination substitutions selected from the group consisting of 19D40D, 36D61R, 36D65L, 40D61R, 40D65Y, 40G65Y, 81K91D, where the residue position (i.e., 19, 36, 40, 61, 65, 81, or 91) is relative to the mature human IL-2 sequence (i.e., relative to SEQ ID NO:4). See Example 2, and Table 3 herein below. Human IL-2 muteins within subclass (2) of the first functional class include those muteins comprising the amino acid sequence of des-alanyl-1, C125S human IL-2 (SEQ ID NO:8) or C125S human IL-2 (SEQ ID NO:6) with at least one of the combination substitutions selected from the group consisting of 40D72N, 80K65Y, 81K88D, 81K42E, 81K72N, 107H65Y, 107R72N, where the residue position (i.e., 40, 42, 65, 72, 80, 81, 88, or 107) is relative to the mature human IL-2 sequence (i.e., relative to SEQ ID NO:4). See Example 2, and Table 4 herein below.

The second functional class of human IL-2 muteins includes those muteins that strongly increase NK cell proliferation without deleterious impact on IL-2-induced TNF-α production by NK cells. Muteins within this functional group meet three selection criteria: (1) level of IL-2-induced NK cell proliferation that is greater than about 200% of that induced by des-alanyl-1, C125S human IL-2 or C125S human IL-2 at one or more concentrations of human IL-2 mutein selected from the group consisting of 0.005 nM (i.e., 5 pM), 0.02 nM (i.e., 20 pM), 0.05 nM (i.e., 50 pM), 0.1 nM (i.e., 100 pM), or 1.0 nM (i.e., 1000 pM); (2) level of IL-2-induced NK cell proliferation that is greater than about 150% of that induced by des-alanyl-1, C125S human IL-2 or C125S human IL-2 when measured for at least two concentrations of human IL-2 mutein selected from the group consisting of 0.005 nM (i.e., 5 pM), 0.02 nM (i.e., 20 pM), 0.05 nM (i.e., 50 pM), 0.1 nM (i.e., 100 pM), or 1.0 nM (i.e., 1000 pM); and (3) a level of IL-2-induced TNF-α production by NK cells that is similar to (i.e., ±10%) that induced by the reference IL-2 muteins or, preferably, less than 90% of that induced by the reference IL-2 muteins, where TNF-α production is assayed at a mutein concentration of 1.0 nM (i.e., 1000 pM) or 0.1 nM (i.e., 100 pM). In one embodiment, IL-2-induced TNF-α production by NK cells and IL-2-induced NK cell proliferation are determined using NK-92 cells (i.e., using the NK-92 bioassay disclosed herein), in which TNF-α production is measured using ELISA, and NK cell proliferation is measured by an MTT assay as noted herein above. Human IL-2 muteins within this second functional class include those muteins comprising the amino acid sequence of des-alanyl-1, C125S human IL-2 (SEQ ID NO:8) or C125S human IL-2 (SEQ ID NO:6) with at least one of the combination substitutions selected from the group consisting of 19D81K, 40G36D, and 81K36D, where the residue position (i.e., 19, 36, 40, or 81) is relative to the mature human IL-2 sequence (i.e., relative to SEQ ID NO:4). See Example 3, and Table 5 herein below.

The third functional class of human IL-2 muteins includes those muteins that are “bi-functional” in that they induce increased NK cell proliferation and decreased TNF-α production by NK cells relative to the reference IL-2 muteins. Muteins within this third functional class meet the following criteria: (1) induce a level of NK cell proliferation that is at least about 150% of that observed for des-alanyl-1, C125S human IL-2 or C125S human IL-2 when assayed for any one mutein concentration selected from the group consisting of 0.005 nM (i.e., 5 pM), 0.02 nM (i.e., 20 pM), 0.05 nM (i.e., 50 pM), 0.1 nM (i.e., 100 pM), or 1.0 nM (i.e., 1000 pM); and (2) induce a level of TNF-α production by NK cells that is less than about 75% of that induced by des-alanyl-1, C125S human IL-2 or C125S human IL-2 when assayed at a mutein concentration of about 1.0 nM. In one embodiment, IL-2-induced TNF-α production and IL-2-induced NK cell proliferation are determined using NK-92 cells (i.e., the NK-92 bioassay disclosed herein), in which IL-2-induced TNF-α production is measured using ELISA, and IL-2-induced NK cell proliferation is measured by an MTT assay as noted herein above. Human IL-2 muteins within this third functional class include those muteins comprising the amino acid sequence of des-alanyl-1, C125S human IL-2 (SEQ ID NO:8) or C125S human IL-2 (SEQ ID NO:6) with at least one of the combination substitutions selected from the group consisting of 36D42R, 36D80K, 40D80K, 81K61R, 91N95G, 107H36D, 107R36D, and 91N94Y95G, where the residue position (i.e., 36, 40, 42, 61, 80, 81, 91, 94, 95, or 107) is relative to the mature human IL-2 sequence (i.e., relative to SEQ ID NO:4). See Example 4, and Table 6 herein below.

The present invention also provides human IL-2 muteins meeting other selection criteria that contribute to an improved therapeutic index relative to that observed for desalanyl-1, C125S human IL-2 or C125S human IL-2. Thus, for example, in another embodiment, the present invention provides human IL-2 muteins that also exhibit a ratio of IL-2-induced NK cell proliferation at 0.1 nM mutein relative to IL-2-induced TNF-α production by NK cells at 1.0 nM mutein that is at least 1.25-fold greater, 1.5-fold greater, 1.75-fold greater, preferably at least 2.0-fold greater, 2.5-fold greater, 3.0-fold greater, more preferably at least 3.5-fold greater, 3.75-fold greater, 4.0-fold greater, 4.5-fold greater, and up to about 5.0-fold greater than that observed for des-alanyl-1, C125S human IL-2 or C125S human IL-2. Muteins meeting these criteria include all of the muteins shown in Table 1, with the exception of the human IL-2 mutein comprising the 19D40D combination substitution. An increase in this index is predictive of improved clinical benefit in view of the beneficial effects of enhanced NK cell effector function and reduced toxicity.

Biologically Active Variants of Novel Human IL-2 Muteins

The present invention also provides biologically active variants of the novel human IL-2 muteins disclosed herein that also have these improved properties relative to the reference IL-2 molecule, i.e., the biologically active variants induce low or reduced pro-inflammatory cytokine production by NK cells, as well as maintain or increase NK cell proliferation, when compared to the reference IL-2 molecule, i.e., des-alanyl-1, C125S or C125S human IL-2, using the bioassays disclosed elsewhere herein. As noted previously, it is recognized that a variant of any given novel human IL-2 mutein identified herein may have a different absolute level of a particular biological activity relative to that observed for the novel human IL-2 mutein of the invention, so long as it has the desired characteristics relative to the reference IL-2 molecules, i.e., reduced toxicity, that is reduced pro-inflammatory cytokine production, and/or increased NK cell proliferation when compared to the reference IL-2 molecule, i.e., des-alanyl-1, C125S human IL-2 or C125S human IL-2, using the bioassays disclosed elsewhere herein.

By “variant” is intended substantially similar sequences. Variants of the novel human IL-2 muteins described herein may be derived from naturally occurring (e.g., allelic variants that occur at the IL-2 locus) or recombinantly produced (for example muteins) nucleic acid or amino acid sequences. Polypeptide variants can be fragments of the novel human IL-2 muteins disclosed herein, or they can differ from the novel human IL-2 muteins by having one or more additional amino acid substitutions or deletions, or amino acid insertions, so long as the variant polypeptide retains the particular amino acid substitutions of interest that are present within the novel human IL-2 muteins disclosed herein. Thus, suitable polypeptide variants include those with the C125S substitution corresponding to position 125 of the mature human IL-2 sequence (i.e., SEQ ID NO:4), one of the combination substitutions identified herein as contributing to the improved therapeutic index of the novel human IL-2 muteins of the present invention (i.e., the combination substitutions shown in Table 1 above), and which have one or more additional amino acid substitutions or deletions, or amino acid insertions. Thus, for example, where the novel human IL-2 mutein comprises the amino acid sequence of desalanyl-1, C125S human IL-2 (SEQ ID NO:8) or C125S human IL-2 (SEQ ID NO:6) with one of the combination substitutions shown in Table 1, suitable biologically active variants of these novel human IL-2 muteins will also comprise the C125S substitution as well as the combination substitution shown in Table 1, but can differ from the respective novel human IL-2 mutein in having one or more additional substitutions, insertions, or deletions, so long as the variant polypeptide has the desired characteristics relative to the reference IL-2 molecule (i.e., the reference IL-2 mutein C125S human IL-2 or des-alanyl-1, C125S human IL-2), and thus has reduced toxicity, that is reduced pro-inflammatory cytokine production, and/or increased NK cell proliferation when compared to the reference IL-2 molecule (i.e., C125S human IL-2 or des-alanyl-1, C125S human IL-2). Such variants will have amino acid sequences that are at least 70%, generally at least 75%, 80%, 85%, 90% identical, preferably at least 91%, 92%, 93%, 94%, 95%, 96%, 97%, 98%, 99% identical to the amino acid sequence for the respective novel human IL-2 mutein, for example, the novel human IL-2 mutein set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72, where percent sequence identity is determined as noted herein below.

In other embodiments, the biologically active variants will have amino acid sequences that are at least 70%, generally at least 75%, 80%, 85%, 90% identical, preferably at least 91%, 92%, 93%, 94%, 95%, 96%, 97%, 98%, 99% identical to the amino acid sequence set forth in residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72, where percent sequence identity is determined as noted herein below.

In some embodiments of the invention, biologically active variants of the human IL-2 muteins of the invention have the C125S substitution replaced with another neutral amino acid such as alanine, which does not affect the desired functional characteristics of the human IL-2 mutein. Thus, for example, such variants have an amino acid sequence that comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72. In yet other embodiments, the biologically active variants of the human IL-2 muteins of the invention comprise residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72, with the exception of having an alanine residue substituted for the serine residue at position 125 of these sequences.

In alternative embodiments of the invention, biologically active variants of the human IL-2 muteins of the invention comprise the amino acid sequence of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72, with the exception of having a cysteine residue substituted for the serine residue at position 125 of these sequences. In yet other embodiments, the biologically active variants of the human IL-2 muteins of the invention comprise residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72, with the exception of having a cysteine residue substituted for the serine residue at position 125 of these sequences.

By nucleic acid “variant” is intended a polynucleotide that encodes a novel human IL-2 mutein of the invention but whose nucleotide sequence differs from the novel mutein sequence disclosed herein due to the degeneracy of the genetic code. Codons for the naturally occurring amino acids are well known in the art, including those codons that are most frequently used in particular host organisms used to express recombinant proteins. The nucleotide sequences encoding the IL-2 muteins disclosed herein include those set forth in the accompanying Sequence Listing, as well as nucleotide sequences that differ from the disclosed sequences because of degeneracy in the genetic code.

Thus, for example, where the IL-2 mutein of the invention comprises an aspartic acid (i.e., D) substitution, such as in the C125S or des-alanyl-1, C125S mutein comprising the 19D40D, 19D81K, 36D42R, 36D61R, 36D65L, 40D36D, 40D61R, 40D65Y, 40D72N, 40D80K, 40G36D, 80K36D, 81K36D, 81K88D, 81K91D, 107H36D, 107R36D, or 40D81K107H combination substitution, the nucleotide sequence encoding the substituted aspartic acid residue can be selected from the two universal triplet codons for aspartic acid, i.e., GAC and GAT. Where the combination substitution comprises two similar residue substitutions, such as in the muteins comprising the 19D40D or 40D36D combination substitution, the substituted residues can be encoded by the same universal codon (i.e., both substitutions encoded by either GAC or GAT) or can be encoded by alternative universal codons (i.e., one substitution encoded by GAC and the other substitution encoded by GAT). Similarly, where the IL-2 mutein of the invention comprises a glycine (i.e., G) substitution, such as in the C125S or des-alanyl-1, C125S mutein comprising the 40G36D, 40G65Y, 91N95G, 40G81K107H, or 91N94Y95G combination substitution, the nucleotide sequence encoding the substituted glycine residue can be selected from the four universal triplet codons for glycine, i.e., GGT, GGC, GGA, and GGG.

Where the IL-2 mutein of the invention comprises a lysine (i.e., K) substitution, such as in the C125S or des-alanyl-1, C125S mutein comprising the 19D81K, 40D80K, 80K36D, 80K65Y, 81K36D, 81K42E, 81K61R, 81K65Y, 81K72N, 81K38D, 81K91D, 81K107H, 40D81K107H, or 40G81K107H combination substitution, the nucleotide sequence encoding the substituted lysine residue can be selected from the two universal triplet codons for lysine, i.e., AAA and AAG. Similarly, where the IL-2 mutein of the invention comprises a leucine (i.e., L) substitution, such as in the C125S or des-alanyl-1, C125S mutein comprising the 36D65L or 8IL107H combination substitution, the nucleotide sequence encoding the substituted leucine residue can be selected from the six universal triplet codons for leucine, i.e., TTA, TTG, CTT, CTC, CTA, and CTG.

Where the IL-2 mutein of the invention comprises an asparagine (i.e., N) substitution, such as in the C125S or des-alanyl-1, C125S mutein comprising the 40D72N, 81K72N, 91N95G, 107R72N, or 91N94Y95G combination substitution, the nucleotide sequence encoding the substituted asparagine residue can be selected from the two universal triplet codons for asparagine, i.e., GAT and GAC. Similarly, where the IL-2 mutein of the invention comprises a histidine (i.e., H) substitution, such as in the C125S or des-alanyl-1, C125S mutein comprising the 81K107H, 81L107H, 107H36D, 107H42E, 107H65Y, 40D81K107H, or 40G81K107H combination substitution, the nucleotide sequence encoding the substituted histidine residue can be selected from the two universal triplet codons for histidine, i.e., CAT and CAC.

Where the IL-2 mutein of the invention comprises an arginine (i.e., R) substitution, such as in the C125S or des-alanyl-1, C125S mutein comprising the 36D42R, 36D61R, 40D61R, 81K61R, 107R36D, or 107R72N combination substitution, the nucleotide sequence encoding the substituted arginine residue can be selected from the six universal triplet codons for arginine, i.e., CGT, CGC, CGA, CGG, AGA, and AGG. Similarly, where the IL-2 mutein of the invention comprises a tyrosine (i.e., Y) substitution, such as in the C125S or des-alanyl-1, C125S mutein comprising the 40D65Y, 40G65Y, 80K65Y, 81K65Y, 107H65Y, or 91N94Y95G combination substitution, the nucleotide sequence encoding the substituted tyrosine residue can be selected from the two universal triplet codons for tyrosine, i.e., TAT and TAC. Where the IL-2 mutein of the invention comprises a glutamic acid (i.e., E) substitution, such as in the C125S or des-alanyl-1, C125S mutein comprising the 81K42E or 107H42E combination substitution, the nucleotide sequence encoding the substituted glutamic acid residue can be selected from the two universal triplet codons for glutamic acid, i.e., GAA and GAG.

Though the foregoing list of nucleic acid variants have recited the universal codons that could be utilized to encode the particular residue substitutions identified therein, it is recognized that the present invention encompasses all nucleic acid variants that encode the human IL-2 muteins disclosed herein as a result of degeneracy in the genetic code.

Naturally occurring allelic variants of native human IL-2 can be identified with the use of well-known molecular biology techniques, such as polymerase chain reaction (PCR) and hybridization techniques, and can serve as guidance to the additional mutations that can be introduced into the human IL-2 muteins disclosed herein without impacting the desired therapeutic index of these novel human IL-2 muteins. Variant nucleotide sequences also include muteins derived from synthetically derived nucleotide sequences that have been generated, for example, by site-directed mutagenesis but which still encode the novel IL-2 muteins disclosed herein, as discussed below. Generally, nucleotide sequence variants of the invention will have 70%, generally at least 75%, 80%, 85%, 90% sequence identity, preferably at least 91%, 92%, 93%, 94%, 95%, 96%, 97%, 98%, or 99% sequence identity to their respective novel human IL-2 mutein nucleotide sequences, for example, with respect to a novel human IL-2 mutein coding sequence set forth in SEQ ID NO: 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71, where percent sequence identity is determined as noted herein below. In other embodiments, nucleotide sequence variants of the invention will have at least 70%, generally at least 75%, 80%, 85%, 90% sequence identity, preferably at least 91%, 92%, 93%, 94%, 95%, 96%, 97%, 98%, or 99% sequence identity to nucleotides 4-399 of the coding sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71, where percent sequence identity is determined as noted herein below.

As used herein, the terms “gene” and “recombinant gene” refer to nucleic acid molecules comprising an open reading frame encoding an IL-2 mutein of the invention. As used herein, the phrase “allelic variant” refers to a nucleotide sequence that occurs at an IL-2 locus or to a polypeptide encoded by that nucleotide sequence. Such natural allelic variations can typically result in 1-5% variance in the nucleotide sequence of the IL-2 gene. Any and all such nucleotide variations and resulting amino acid polymorphisms or variations in a IL-2 sequence that are the result of natural allelic variation and that do not alter the functional activity of the novel human IL-2 muteins of the invention are intended to be sequences which can be mutated according to the present invention, and all of the resulting sequences are intended to fall within the scope of the invention.

For example, amino acid sequence variants of the novel human IL-2 muteins disclosed herein can be prepared by making mutations in the cloned DNA sequence encoding the novel IL-2 mutein, so long as the mutation(s) does not alter the combination substitutions identified in Table 1. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (2d ed., Cold Spring Harbor Laboratory Press, Plainview, N.Y.); U.S. Pat. No. 4,873,192; and the references cited therein; herein incorporated by reference. Guidance as to appropriate amino acid substitutions that may not affect the desired biological activity of the IL-2 mutein (i.e., reduced pro-inflammatory production by NK cells predictive of reduced toxicity and maintained or increased NK cell proliferation) may be found in the model of Dayhoff et al. (1978) Atlas of Polypeptide Sequence and Structure (Natl. Biomed. Res. Found., Washington, D.C.), herein incorporated by reference.

When designing biologically active variants of a human IL-2 mutein disclosed herein, conservative substitutions, such as exchanging one amino acid with another having similar properties, may be preferred. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). See, for example, Bowie et al. (1990) Science 247:1306, herein incorporated by reference. Examples of conservative substitutions include, but are not limited to, Gly←÷Ala, Val←÷Ile←÷Leu, Asp←÷Glu, Lys←÷Arg, Asn←÷Gln, and Phe←÷Trp←÷Tyr. Preferably, such substitutions would not be made for conserved cysteine residues, such as the amino terminal contiguous cysteine residues.

Guidance as to regions of the human IL-2 protein that can be altered either via residue substitutions, deletions, or insertions outside of the desired substitutions identified herein can be found in the art. See, for example, the structure/function relationships and/or binding studies discussed in Bazan (1992) Science 257:410-412; McKay (1992) Science 257:412; Theze et al. (1996) Immunol. Today 17:481-486; Buchli and Ciardelli (1993) Biochem. Biophys 307:411-415;Collins et al. (1988) Proc. Natl. Acad. Sci. USA 85:7709-7713; Kuziel et al. (1993) J. Immunol. 150:5731; Eckenberg et al. (1997) Cytokine 9:488-498; the contents of which are herein incorporated by reference in their entirety.

In constructing variants of a novel human IL-2 mutein of the invention, modifications to the nucleotide sequences encoding the variants will be made such that variant polypeptides may continue to possess the desired activity. Obviously, any mutations made in the DNA encoding a variant polypeptide must not place the sequence out of reading frame and preferably will not create complementary regions that could produce secondary mRNA structure. A variant of a polypeptide may differ by as few as 1 to 15 amino acid residues, such as 6-10, as few as 5, as few as 4, 3, 2, or even 1 amino acid residue. A variant of a nucleotide sequence may differ by as few as 1 to 30 nucleotides, such as 6 to 25, as few as 5, as few as 4, 3, 2, or even 1 nucleotide.

Biologically active variants of the human IL-2 muteins of the invention include fragments of these muteins. By “fragment” is intended a portion of the coding nucleotide sequence or a portion of the amino acid sequence. With respect to coding sequences, fragments of a human IL-2 mutein nucleotide sequence may encode mutein fragments that retain the desired biological activity of the novel human IL-2 mutein. A fragment of a novel human IL-2 mutein disclosed herein may be 15, 20, 25, 30, 35, 40, 45, 50, 55, 60, 65, 70, 75, 80, 85, 90, 95, 100, 105, 110, 115, 120, 125, 130 amino acids or up to the full length of the novel human IL-2 polypeptide. Fragments of a coding nucleotide sequence may range from at least 45, 60, 75, 90, 105, 120, 135, 150, 165, 180, 195, 210, 225, 240, 255, 270, 285, 300, 315, 330, 345, 360, 375, 390, nucleotides, and up to the entire nucleotide sequence encoding the novel human IL-2 mutein.

The human IL-2 muteins disclosed herein and biologically active variants thereof may be modified further so long as they have the desired characteristics relative to the reference IL-2 molecules, ie., reduced toxicity and/or increased NK cell proliferation relative to the C125S human IL-2 mutein or des-alanyl-1, C125S human IL-2 mutein. Further modifications include, but are not limited to, phosphorylation, substitution of non-natural amino acid analogues, and the like. Modifications to IL-2 muteins that may lead to prolonged in vivo exposure, and hence increase efficacy of the IL-2 mutein pharmaceutical formulations, include glycosylation or PEGylation of the protein molecule. Glycosylation of proteins not natively glycosylated is usually performed by insertion of N-linked glycosylation sites into the molecule. This approach can be used to prolong half-life of proteins such as IL-2 muteins. In addition, this approach can be used to shield immunogenic epitopes, increase protein solubility, reduce aggregation, and increase expression and purification yields.

Once the variants of the human IL-2 muteins disclosed herein are obtained, the deletions, insertions, and substitutions of the human IL-2 mutein sequences are not expected to produce radical changes in the characteristics of the particular human IL-2 mutein. However, when it is difficult to predict the exact effect of the substitution, deletion, or insertion in advance of doing so, one skilled in the art will appreciate that the effect will be evaluated by routine screening assays. That is, the IL-2-induced NK or T cell proliferation activity can be evaluated by standard cell proliferation assays known to those skilled in the art, including the assays described herein. IL-2-induced pro-inflammatory cytokine production may be measured using cytokine-specific ELISAs, for example, the TNF-α specific ELISA noted elsewhere herein. NK cell survival signaling may be measured by a pAKT ELISA (see, for example, the assay described herein below). NK cell-mediated cytolytic activity (i.e., cytotoxicity) may be measured by assays known in the art (for example, measurement of NK-mediated, LAK-mediated, or ADCC-mediated cytolytic activity as noted elsewhere herein).

The human IL-2 muteins disclosed herein, and biologically active variants thereof, can be constructed as IL-2 fusions or conjugates comprising the IL-2 mutein (or biologically active variant thereof as defined herein) fused to a second protein or covalently conjugated to polyproline or a water-soluble polymer to reduce dosing frequencies or to further improve IL-2 tolerability. For example, the human IL-2 mutein (or biologically active variant thereof as defined herein) can be fused to human albumin or an albumin fragment using methods known in the art (see, for example, WO 01/79258). Alternatively, the human IL-2 mutein (or biologically active variant thereof as defined herein) can be covalently conjugated to polyproline or polyethylene glycol homopolymers and polyoxyethylated polyols, wherein the homopolymer is unsubstituted or substituted at one end with an alkyl group and the poplyol is unsubstituted, using methods known in the art (see, for example, U.S. Pat. Nos. 4,766,106, 5,206,344, and 4,894,226).

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5: Suppl. 3 (National Biomedical Research Foundation, Washington, D.C.). With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences can be accomplished using a mathematical algorithm. For purposes of the present invention, percent sequence identity of an amino acid sequence is determined using the Smith-Waterman homology search algorithm using an affine 6 gap search with a gap open penalty of 12 and a gap extension penalty of 2, BLOSUM matrix 62. The Smith-Waterman homology search algorithm is taught in Smith and Waterman (1981) Adv. Appl. Math 2:482-489, herein incorporated by reference. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

It is further recognized that when considering percentage of amino acid identity, some amino acid positions may differ as a result of conservative amino acid substitutions, which do not affect properties of polynucleotide function. In these instances, percent sequence identity may be adjusted upwards to account for the similarity in conservatively substituted amino acids. Such adjustments are well known in the art. See, for example, Meyers et al. (1988) Computer Applic. Biol. Sci. 4:11-17.

Recombinant Expression Vectors and Host Cells

Generally, the human IL-2 muteins of the invention will be expressed from vectors, preferably expression vectors. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of coding sequences to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses).

The expression constructs or vectors of the invention comprise a nucleic acid molecule encoding a human IL-2 mutein of the present invention in a form suitable for expression of the nucleic acid molecule in a host cell. The coding sequence of interest can be prepared by recombinant DNA techniques as described, for example, by Taniguchi et al. (1983) Nature 302:305-310 and Devos (1983) Nucleic Acids Research 11:4307-4323 or using mutationally altered IL-2 as described by Wang et al. (1984) Science 224:1431-1433. It is recognized that the coding sequences set forth in odd SEQ ID NOS:9-71 begin with a codon for the first residue of the mature human IL-2 sequence of SEQ ID NO:4 (i.e., a codon for the alanine at position 1), rather than a codon for methionine, which generally is the translation initiation codon ATG in messenger RNA. These disclosed nucleotide sequences also lack a translation termination codon following the nucleotide at position 399 of odd SEQ ID NOS:9-71. Where these sequences, or sequences comprising nucleotides 4-399 of odd SEQ ID NOS:9-71, are to be used to express the human IL-2 muteins of the invention, it is recognized that the expression construct comprising these human IL-2 mutein coding sequences will further comprise a translation initiation codon, for example, an ATG codon, upstream and in proper reading frame with the human IL-2 mutein coding sequence. The translation initiation codon can be provided at an upstream location from the initial codon of the human IL-2 mutein coding sequence by utilizing a translation initiation codon, for example ATG, that is already in a sequence that comprises the human IL-2 mutein coding sequence, or can otherwise be provided from an extraneous source such as the plasmid to be used for expression, providing that the translation initiation codon first appearing before the initial codon in the human IL-2 mutein coding sequence is in proper reading frame with the initial codon in the human IL-2 mutein coding sequence. Similarly, the human IL-2 mutein coding sequence disclosed herein will be followed by one or more translation termination codons, for example, TGA, to allow for production of a human IL-2 mutein that ends with the last amino acid of the sequence set forth in even SEQ ID NOS: 10-72.

The recombinant expression vectors include one or more regulatory sequences, selected on the basis of the host cells to be used for expression, operably linked to the nucleic acid sequence to be expressed. “Operably linked” is intended to mean that the nucleotide sequence of interest (i.e., a sequence encoding a human IL-2 mutein of the present invention) is linked to the regulatory sequence(s) in a manner that allows for expression of the nucleotide sequence (e.g., in an in vitro transcription/translation system or in a host cell when the vector is introduced into the host cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression constructs of the invention can be introduced into host cells to thereby produce the human IL-2 muteins disclosed herein or to produce biologically active variants thereof.

The expression constructs or vectors of the invention can be designed for expression of the human IL-2 mutein or variant thereof in prokaryotic or eukaryotic host cells. Expression of proteins in prokaryotes is most often carried out in Escherichia coli with vectors containing constitutive or inducible promoters. Strategies to maximize recombinant protein expression in E. coli can be found, for example, in Gottesman (1990) in Gene Expression Technology: Methods in Enzymology 185 (Academic Press, San Diego, Calif.), pp. 119-128 and Wada et al. (1992) Nucleic Acids Res. 20:2111-2118. Processes for growing, harvesting, disrupting, or extracting the human IL-2 mutein or variant thereof from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

The recombinant human IL-2 muteins or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.).

The sequences encoding the human IL-2 muteins of the present invention can be optimized for expression in the host cell of interest. The G-C content of the sequence may be adjusted to levels average for a given cellular host, as calculated by reference to known genes expressed in the host cell. Methods for codon optimization are well known in the art. Individual codons can be optimized, for example, the codons where residue substitutions have been made, for example, the C125S substitution, the C125A substitution, and/or the additional combination substitution indicated in Table 1. Alternatively, other codons within the human IL-2 mutein coding sequence can be optimized to enhance expression in the host cell, such that 1%, 5%, 10%, 25%, 50%, 75%, or up to 100% of the codons within the coding sequence have been optimized for expression in a particular host cell. See, for example, the human IL-2 mutein sequences disclosed in SEQ ID NOS:73 and 74, where the codons for the 36D61R and 107R36D combination substitutions, respectively, have been optimized for expression in E. coli.

The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, particle gun, or electroporation. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

Prokaryotic and eukaryotic cells used to produce the IL-2 muteins of this invention and biologically active variants thereof are cultured in suitable media, as described generally in Sambrook et al. (1989) Molecular Cloning: A Laboratory Manual (2d ed., Cold Spring Harbor Laboratory Press, Plainview, N.Y.).

Pharmaceutical Compositions

After the human IL-2 muteins or variants thereof are produced and purified, they may be incorporated into a pharmaceutical composition for application in human and veterinary therapeutics, such as cancer therapy, immunotherapy, and the treatment of infectious diseases. Thus, the human IL-2 muteins or biologically active variants thereof can be formulated as pharmaceutical formulations for a variety of therapeutic uses. As a composition, the human IL-2 muteins or biologically active variants thereof are parenterally administered to the subject by methods known in the art. Subjects include mammals, e.g., primates, humans, dogs, cattle, horses, etc. These pharmaceutical compositions may contain other compounds that increase the effectiveness or promote the desirable qualities of the human IL-2 muteins of the invention. The pharmaceutical compositions must be safe for administration via the route that is chosen, they must be sterile, retain bioactivity, and they must stably solubilize the human IL-2 mutein or biologically active variant thereof. Depending upon the formulation process, the IL-2 mutein pharmaceutical compositions of the invention can be stored in liquid form either ambient, refrigerated, or frozen, or prepared in the dried form, such as a lyophilized powder, which can be reconstituted into the liquid solution, suspension, or emulsion before administration by any of various methods including oral or parenteral routes of administration.

Such pharmaceutical compositions typically comprise at least one human IL-2 mutein, biologically active variant thereof, or a combination thereof, and a pharmaceutically acceptable carrier. Methods for formulating the human IL-2 muteins of the invention for pharmaceutical administration are known to those of skill in the art. See, for example, Gennaro (ed.) (1995) Remington: The Science and Practice of Pharmacy (19^{th} ed., Mack Publishing Company, Easton, Pa.).

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the human IL-2 mutein pharmaceutical formulations of the invention. Supplementary active compounds can also be incorporated into the compositions.

An IL-2 mutein pharmaceutical composition comprising a human IL-2 mutein of the invention or variant thereof is formulated to be compatible with its intended route of administration. The route of administration will vary depending on the desired outcome. The IL-2 mutein pharmaceutical composition can be administered by bolus dose, continuous infusion, or constant infusion (infusion for a short period of time, i.e. 1-6 hours). The IL-2 mutein pharmaceutical composition can be administered orally, intranasally, parenterally, including intravenously, subcutaneously, intraperitoneally, intramuscularly, etc., by intradermal, transdermal (topical), transmucosal, and rectal administration, or by pulmonary inhalation.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; surfactants such as polysorbate 80; SDS; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. Where formation of protein aggregates is minimized in the formulation process, suitable carriers for intravenous administration include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a protein or antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The human IL-2 muteins of the present invention, or biologically active variants thereof, can be formulated using any known formulation process known in the art for human IL-2. Suitable formulations that are useful in the present method are shown in various patents and publications. For example, U.S. Pat. No. 4,604,377 shows a preferred IL-2 formulation that has a therapeutic amount of IL-2, which is substantially free from non-IL-2 protein and endotoxin, a physiologically acceptable water-soluble carrier, and a sufficient amount of a surface active agent to solubilize the IL-2, such as sodium dodecyl sulfate. Other ingredients can be included, such as sugars. U.S. Pat. No. 4,766,106 shows formulations including polyethylene glycol (PEG) modified IL-2. European patent application, Publication No. 268,110, shows IL-2 formulated with various non-ionic surfactants selected from the group consisting of polyoxyethylene sorbitan fatty acid esters (Tween-80), polyethylene glycol monostearate, and octylphenoxy polyethoxy ethanol compounds (Triton X405). U.S. Pat. No. 4,992,271 discloses IL-2 formulations comprising human serum albumin and U.S. Pat. No. 5,078,997 discloses IL-2 formulations comprising human serum albumin and amino acids. U.S. Pat. No. 6,525,102 discloses IL-2 formulations comprising an amino acid base, which serves as the primary stabilizing agent of the polypeptide, and an acid and/or its salt form to buffer the solution within an acceptable pH range for stability of the polypeptide. Copending U.S. patent application Ser. No. 10/408,648 discloses IL-2 formulations suitable for pulmonary delivery. All of the above patents and patent applications are hereby incorporated by reference in their entireties.

Therapeutic Uses

Pharmaceutical formulations comprising the human IL-2 muteins of the present invention or biologically active variants thereof obtained from these human IL-2 muteins are useful in the stimulation of the immune system, and in the treatment of cancers, such as those currently treated using native human IL-2 or Proleukin® IL-2. The human IL-2 muteins of the present invention and suitable biologically active variants thereof have the advantage of reducing pro-inflammatory cytokine production predictive of having lower toxicity, while maintaining or enhancing desirable functional activities such as NK cell proliferation, survival, NK-mediated cytotoxicity (NK, LAK, and ADCC), and T cell proliferation.

Because of the predicted lower toxicity, in those clinical indications requiring high doses of IL-2, the human IL-2 muteins of the present invention, and biologically active variants thereof, can be administered at similar or higher doses than can native IL-2 or Proleukin® IL-2 while minimizing toxicity effects. Thus, the present invention provides a method for reducing interleukin-2 (IL-2)-induced toxicity symptoms in a subject undergoing IL-2 administration as a treatment protocol, where the method comprising administering the IL-2 as an IL-2 mutein disclosed herein. Furthermore, the human IL-2 muteins of the present invention and suitable biologically active variants thereof have the additional advantage of greater therapeutic efficacy, so that lower doses of these human IL-2 muteins can provide greater therapeutic efficacy than comparable doses of native IL-2 or Proleukin® IL-2.

A pharmaceutically effective amount of an IL-2 mutein pharmaceutical composition of the invention is administered to a subject. By “pharmaceutically effective amount” is intended an amount that is useful in the treatment, prevention or diagnosis of a disease or condition. By “subject” is intended mammals, e.g., primates, humans, dogs, cats, cattle, horses, pigs, sheep, and the like. Preferably the subject undergoing treatment with the pharmaceutical formulations of the invention is human.

When administration is for the purpose of treatment, administration may be for either a prophylactic or therapeutic purpose. When provided prophylactically, the substance is provided in advance of any symptom. The prophylactic administration of the substance serves to prevent or attenuate any subsequent symptom. When provided therapeutically, the substance is provided at (or shortly after) the onset of a symptom. The therapeutic administration of the substance serves to attenuate any actual symptom.

Thus, for example, formulations comprising an effective amount of a pharmaceutical composition of the invention comprising a human IL-2 mutein of the invention or biologically active variant thereof can be used for the purpose of treatment, prevention, and diagnosis of a number of clinical indications responsive to therapy with IL-2. The human IL-2 muteins of the present invention and biologically active variants thereof can be formulated and used in the same therapies as native-sequence IL-2 or Proleukin® IL-2. Accordingly, formulations of the invention comprising a human IL-2 mutein of the invention or biologically active variant thereof are useful for the diagnosis, prevention, and treatment (local or systemic) of bacterial, viral, parasitic, protozoan and fungal infections; for augmenting cell-mediated cytotoxicity; for stimulating lymphokine activated killer (LAK) cell activity; for mediating recovery of immune function of lymphocytes; for augmenting alloantigen responsiveness; for facilitating immune reconstitution in cancer patients following radiotherapy, or following or in conjunction with bone marrow or autologous stem cell transplantation; for facilitating recovery of immune function in acquired immune deficient states; for reconstitution of normal immunofunction in aged humans and animals; in the development of diagnostic assays such as those employing enzyme amplification, radiolabelling, radioimaging, and other methods known in the art for monitoring IL-2 levels in the diseased state; for the promotion of T-cell growth in vitro for therapeutic and diagnostic purposes; for blocking receptor sites for lymphokines; and in various other therapeutic, diagnostic and research applications. The various therapeutic and diagnostic applications of human IL-2 or variants thereof, such as IL-2 muteins, have been investigated and reported in Rosenberg et al. (1987) N. Engl. J. Med. 316:889-897; Rosenberg (1988) Ann. Surg. 208:121-135; Topalian et al. 1988) J. Clin. Oncol. 6:839-853; Rosenberg et al. (1988) N. Engl. J. Med. 319:1676-1680; Weber et al. (1992) J. Clin. Oncol. 10:33-40; Grimm et al. (1982) Cell. Immunol. 70(2):248-259; Mazumder (1997) Cancer J. Sci. Am. 3(Suppl. 1):S37-42; Mazumder and Rosenberg (1984) J. Exp. Med. 159(2):495-507; and Mazumder et al. (1983) Cancer Immunol. Immunother. 15(1):1-10. Formulations of the invention comprising a human IL-2 mutein of the invention or biologically active variant thereof may be used as the single therapeutically active agent or may be used in combination with other immunologically relevant cells or other therapeutic agents. Examples of relevant cells are B or T cells, NK cells, LAK cells, and the like, and exemplary therapeutic reagents that may be used in combination with IL-2 or variant thereof are the various interferons, especially gamma interferon, B-cell growth factor, IL-1, and antibodies, for example anti-HER2 antibodies such as Herceptin® (Trastuzumab; Genentech, Inc., South San Francisco, Calif.) or anti-CD20 antibodies such as Rituxane (Rituximab; IDEC-C2B8; Biogen IDEC Pharmaceuticals Corp., San Diego, Calif.).

The amount of human IL-2 mutein or biologically active variant thereof administered may range between about 0.1 to about 15 mIU/m². Therapeutically effective doses and particular treatment protocols for IL-2 immunotherapy in combination with anti-cancer monoclonal antibodies are known in the art. See, for example, the doses and treatment protocols disclosed in copending U.S. Patent Application Publication Nos. 2003-0185796, entitled Methods of Therapy for Non-Hodgkin's Lymphoma,” and 20030235556, entitled “Combination IL-2/Anti-HER2 Antibody Therapy for Cancers Characterized by Overexpression of the HER2 Receptor Protein, and copending U.S. Patent Application No. 60/491,371, entitled “Methods of Therapy for Chronic Lymphocytic Leukemia,” Attorney Docket No. 59516-278, filed Jul. 31, 2003; the contents of which are herein incorporated by reference in their entirety. For indications such as renal cell carcinoma and metastatic melanoma, the human IL-2 mutein or biologically active variant thereof may be administered as a high-dose intravenous bolus at 300,000 to 800,000 IU/kg/8hours. See the foregoing U.S. patent applications for recommended doses for IL-2 immunotherapy for B-cell lymphomas, HER2⁺ cancers such as breast cancer, and CLL.

Use of IL-2 immunotherapy for the treatment of HIV infection is also known in the art. See, for example, U.S. Pat. No. 6,579,521, herein incorporated by reference in its entirety, for recommended doses and protocols for this clinical indication.

Thus, the invention provides a method for the treatment of cancer in a subject or for modulating the immune response in a subject, comprising administering a therapeutically effective amount of a human IL-2 mutein of the invention or biologically active variant thereof. The “therapeutically effective amount” refers to a dosage level sufficient to induce a desired biological result without inducing unacceptable toxicity effects. Amounts for administration may vary based upon the concentration of human IL-2 mutein or variant thereof within the pharmaceutical composition, the desired activity, the disease state of the mammal being treated, the dosage form, method of administration, and patient factors such as age, sex, and severity of disease. It is recognized that a therapeutically effective amount is provided in a broad range of concentrations and that the subject may be administered as many therapeutically effective doses as is required to reduce and/or alleviate the signs, symptoms, or causes of the disorder in question, or bring about any other desired alteration of a biological system. Generally, an IL-2 mutein pharmaceutical composition of the invention will comprise the human IL-2 mutein or variant thereof in a concentration range which is greater than that used for Proleukin® IL-2. As the doses are increased relative to that of Proleukin® IL-2, the subject should be closely monitored to determine if toxic side effects appear. Such clinical experimental analyses are well known to those of skill in the art, and would, for example, have been used to established the current doses of Proleukin® IL-2 for use in immunomodulation and cancer therapy.

Bioassays for Monitoring Functional Activity of Human IL-2 Muteins

The present invention also provides novel bioassays for monitoring IL-2 induced NK cell proliferation and TNF-α production, IL-2-induced NK cell-mediated cytotoxicity, IL-2-induced T cell proliferation, and IL-2-induced NK cell survival. These assays have been developed to screen candidate IL-2 muteins for the desired functional profile of reduced pro-inflammatory cytokine production (particularly TNF-α) so as to improve tolerability, and improved NK cell-mediated function as reflected in the ability of the mutein to maintain or increase NK and/or T cell proliferation, to maintain or increase NK-mediated cytotoxicity (NK, LAK, and ADCC), and to maintain or increase NK cell survival.

The first of these assays is referred to herein as the “NK-92 bioassay,” which monitors IL-2 induction of TNF-α production and IL-2-induced NK cell proliferation. This bioassay utilizes the human NK-92 cell line (ATCC CRL-2407, CMCC ID #11925). The NK-92 cell line, originally described by Gong et al. (1994) Leukemia 8(4):652-658, displays phenotypic and functional characteristics of activated NK cells. Proliferation of NK-92 is IL-2 dependent; cells will die if cultured in the absence of IL-2 for 72 hours. The cell line also produces detectable levels of TNF-α within 48-72 hours following exposure to IL-2.

In accordance with the methods of the present invention, candidate IL-2 muteins can be screened for relative ability to induce TNF-α production and induce NK cell proliferation using this NK-92 bioassay. In this manner, NK-92 cells are cultured in complete medium (NK-92 medium) consisting of Alpha-MEM, 12% heat-inactivated fetal bovine serum (FBS), 8% heat-inactivated horse serum, 0.02 mM folic acid, 0.2 mM inositol, 2 mM L-glutamine, and 0.1 mM β-mercaptoethanol. Cultures are seeded at a minimum density of 1-3×10⁵ cells/ml and supplemented with 1000 IU/ml of the reference recombinant human IL-2 mutein (for example, the reference IL-2 mutein designated des-alanyl-1, C125S human IL-2 or the reference C125S human IL-2 mutein). In preparation for the assay, cells are placed in fresh NK-92 medium a minimum of 48 h prior to assay use. One day prior to assay, NK-92 are washed three times and placed in NK-92 medium without any supplemental IL-2 for 24 h. Cells are centrifuged, suspended in NK-92 medium (no IL-2) and plated into 96-well flat bottom plates at a density of 4×10⁴ cells/well in 200 μl with varying concentrations of the reference IL-2 mutein, for example, des-alanyl-1, C125S human IL-2 or C125S human IL-2, or varying concentrations of a candidate IL-2 mutein that is being screened for the functional profile of interest diluted in NK-92 medium. Following a 72-h incubation at 37° C., 5% CO₂, a 100 μl aliquot of culture supernatant is removed and frozen for subsequent quantification of TNF-α using a commercially available TNF-α ELISA kit (for example, BioSource Cytoscreen™ Human TNF-α ELISA kit; Camarillo, Calif.). For the remaining cells in culture, proliferation is determined using a commercially available MTT dye-reduction kit (CeIlTiter 96® Non-Radioactive Cell Proliferation Assay Kit (Promega Corp., Madison, Wis.), and a stimulation index is then calculated based on a colorimetric readout.

The second IL-2 bioassay disclosed herein provides a method for screening candidate IL-2 muteins for their ability to induce natural killer (NK) cell-mediated cytotoxicity. This bioassay, designated the “NK3.3 cytotoxicity bioassay,” utilizes the human NK3.3 cell line. The NK3.3 cell line displays phenotypic and functional characteristics of peripheral blood NK cells (Kombluth (1982) J. Immunol. 129(6):283 1-2837), and can mediate antibody-dependent cellular cytotoxicity (ADCC) via the Fc receptor (CD16, FcγRIIIA). Table 2 in the Experimental section below summarizes the biological activities of NK3.3 cells examined with this IL-2 bioassay.

In accordance with the methods of the present invention, candidate IL-2 muteins can be screened for their cytotoxicity activity using this NK3.3 cytotoxocity bioassay. In this manner, NK3.3 cells are expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for the assay, NK3.3 cells are cultured in the absence of IL-2 (“starved”) for 24 h. The assay consists of 5×10⁴ “starved” NK3.3 cells plated in U-bottom 96-well plates, exposed to varying concentrations of a reference IL-2 mutein, for example, des-alanyl-1, C125S or C125S human IL-2 mutein, or varying concentrations of a candidate IL-2 mutein of interest in a total volume of 100 μl. Following an 18-h incubation, the IL-2-stimulated NK3.3 effector cells are co-incubated with 5×10³ calcein AM-labeled target cells (K562 or Daudi) or antibody-coated, calcein AM-labeled targets (Daudi coated with rituximab at a final concentration of 2 μg/ml) to achieve a final effector-to-target ratio of 10:1 in final volume of 200 μl. Following co-incubation of effector and target cells for 4 h, the 96 well plates are briefly centrifuged; 100 μl of culture supernatant is removed and placed into a black, clear-bottom, flat-bottom 96-well plate for quantitation of calcein AM release by fluorimeter. Quantitation is expressed as percent specific lysis, and is calculated by the following equation: % specific lysis=100×[(mean experimental−mean spontaneous release)/(mean maximal release−mean spontaneous release)]; whereby the spontaneous release is determined from wells containing labeled targets and no effectors, and maximal release is determined from wells containing labeled targets and 1% Triton X-100.

The third IL-2 bioassay disclosed herein provides a method for screening candidate IL-2 muteins for their ability to induce T cell proliferation. In this manner, this IL-2 bioassay for T-cell proliferation utilizes the human T-cell line Kit225 (CMCC ID#11234), derived from a patient with T-cell chronic lymphocytic leukemia (Hori et al. (1987) Blood 70(4):1069-1072). Kit225 cells constitutively express the α, β, γ subunits of the IL-2 receptor complex. Proliferation of Kit225 is IL-2 dependent; cells will die if cultured in the absence of IL-2 for an extended period of time.

In accordance with the present invention, the assay consists of culturing Kit225 cells in the absence of IL-2 for 24 h, followed by plating a specified number of cells with varying concentrations of the reference IL-2 mutein, for example, des-alanyl-1, C125S or C125S human IL-2 mutein, or varying concentrations of a candidate IL-2 mutein of interest. Following a 48-h incubation, proliferation is determined using a standard, commercially available MTT dye reduction kit, and a stimulation index is calculated based on a colorimetric readout.

The fourth IL-2 bioassay of the present invention provides a method for screening candidate IL-2 muteins for their ability to promote NK cell survival. In this manner, candidate muteins are screened for their ability to induce NK cell survival signaling. Proleukin® IL-2 (i.e., the formulation comprising the des-alanyl-1, C125S human IL-2 mutein) induces the phosphorylation of AKT in NK3.3 cells previously starved for IL-2, which is considered a “survival signal.” In accordance with this bioassay, NK3.3 cells are expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for assay, NK3.3 cells are cultured in the absence of IL-2 for 24 h. As an indicator of cell survival signaling, “starved” NK3.3 cells (2×10⁶) are stimulated by addition of 2 nM of the reference IL-2 mutein, for example, the des-alanyl-1, C125S or C125S human IL-2 mutein, or 2 nM of a candidate IL-2 mutein of interest, for 30 min. Cells are washed twice in phosphate buffered saline (PBS). The cell pellet is lysed in 50 μl of a cell extraction buffer containing protease inhibitors and subjected to one freeze-thaw cycle. The extract is centrifuged at 13,000 rpm for 10 min @ 4° C. An aliquot of the cleared lysate is added at a 1:10 dilution to wells of the AKT [pS473]* Inmunoassay Kit (BioSource International). Following the manufacturer's protocol, levels of phosphorylated AKT are detected by quantitative ELISA.

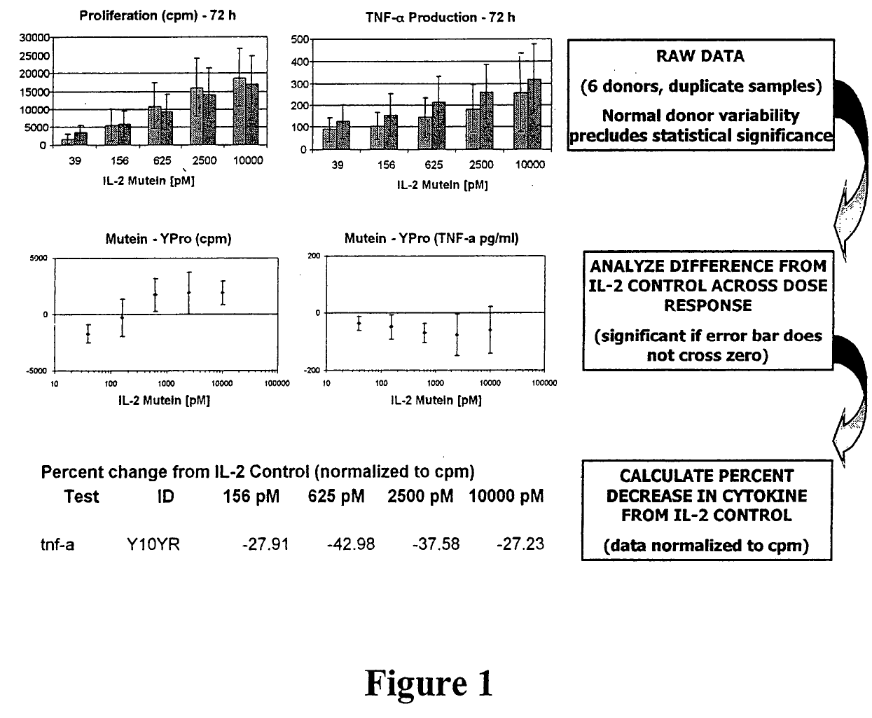

The present invention also provides bioassays for use in screening IL-2 muteins for their functional profiles using human peripheral blood mononuclear cells (PBMC). The first of these bioassays is a combination proliferation/pro-inflammatory cytokine production bioassay. Upon exposure to IL-2, human PBMC proliferate and secrete cytokines in a dose-dependent manner. This combination assay was designed to assess levels of proliferation and cytokine production following 72 h stimulation with a reference IL-2 mutein (such as the des-alanyl-1, C125S mutein or C125S mutein) or a candidate IL-2 mutein of interest. PBMC are isolated by density gradient separation (for example, using ACDA Vacutainer CPT tubes) from one or more normal human donors. In 96-well tissue-culture treated plates, 200,000 cells per well are incubated with various concentrations of IL-2 (0.039 nM-10 nM) or no IL-2 as a negative control in complete RPMI medium (RPMI, 10% heat-inactivated human AB serum, 25 mM HEPES, 2 mM glutamine, penicillin/streptomycin/fungizone) at 37° C., 7% CO₂. Following 66 h of incubation, an aliquot of cell culture supernatant is removed and frozen for cytokine detection at a later time. The cells are pulsed with 1 μCi ³H-thymidine for 6 h, and then harvested to determine levels of nucleotide incorporation (for example, using a Wallac Trilux Microbeta Plate Reader) as a measure of cell proliferation. Commercially available ELISA kits (for example, from BioSource International) can then be used to detect levels of TNF-α in the cell culture supernatants per manufacturer's guidelines. Repeating the assay for a complete panel of separate donors, for example, 6, 8, or 10 donors, provides a characterization of representative proliferative and cytokine responses to IL-2 in a “normal population.” Data can then be analyzed as shown in FIG. 1, and described further herein below in Example 10.

The second PBMC-based bioassay can be used to screen candidate IL-2 muteins for their ability to mediate effector cell cytotoxicity. In this assay, human PBMC are separated from whole blood using density gradient centrifugation. PBMC are stimulated for 3 days in the presence of 10 nM IL-2 control or IL-2 mutein of interest, to generate LAK activity as generally practiced in current state of the art (see for example Isolation of Human NK Cells and Generation of LAK activity IN: Current Protocols in Immunology; 1996 John Wiley & Sons, Inc). The resulting cell population contains “effector” cells, which may be classified as NK or LAK, and can kill K562 and Daudi tumor cell targets, respectively. These effector cells may also mediate ADCC, whereby the effector cells recognize the Fc portion of a specific antibody that is bound to the Daudi target cells. In one embodiment, the antibody bound to the Daudi target cells is Rituxan® (rituximab).

In accordance with the methods of the present invention, human PBMC (effector cells) that have been stimulated with a candidate IL-2 mutein of interest or a reference IL-2 control are co-incubated with calcein AM-labeled target cells at various effector to target cell (E:T ratios) for 4 h. The amount of cytotoxic activity is related to the detection of calcein AM in the culture supernatant. Quantitation is expressed as percent specific lysis at each E:T ratio, based upon determination of spontaneous and maximum release controls. This bioassay examines the following biological activities: natural/spontaneous cytotoxicity (NK), where the target is K562 cells; lymphokine-activated killing (LAK), where the target is Daudi cells; and antibody-dependent cellular cytotoxicity (ADCC), where the target is antibody-coated Daudi cells (for example, Rituxan®-coated Daudi cells).

Data is obtained from a fluorimeter and expressed in relative fluorescence units (rfu). Controls for this bioassay include labeled target cells alone (min) and labeled target cells with final 1% Triton X-100 as a measure of 100% lysis (max). The percent min to max ratio is calculated using the following equation as a measure of assay validity (assay invalid if >30%):

$$\% min to max = 100 ⨯ \frac{mean spontaneous release rfu}{mean maximum release rfu}$$

Once the assay is deemed valid, the mean and standard deviation for triplicate sample points is calculated, followed by the percent specific lysis from mean of triplicate points using the following equation:

$$\% lysis = 100 ⨯ \frac{mean experimental rfu - mean spontaneous release rfu}{mean maximal release rfu - mean spontaneous release rfu}$$

Data is then reported as % specific lysis; in addition, the ratio of candidate IL-2 mutein to relevant IL-2 reference control (for example, des-alanyl-1, C125 S human IL-2 mutein or C125S human IL-2 mutein) can be used to determine whether cytotoxic activity is maintained relative to the IL-2 reference control in a mixed population of human PBMC donors.

The foregoing assays can be utilized to screen candidate IL-2 mutein libraries for desired functional profiles, where the functional activities of interest include one or more of the following: IL-2 induced pro-inflammatory cytokine production (particularly TNF-α and/or IFN-γ), IL-2 induced NK and/or T cell proliferation, IL-2 induced NK-mediated cytotoxicity (NK, LAK, and ADCC), and IL-2 induced NK cell survival.

The following examples are offered by way of illustration and not by way of limitation.

### EXPERIMENTAL

The therapeutic utility of IL-2 is hampered by the toxicities associated with its administration, including fevers, chills, hypotension, and vascular leak syndrome. IL-2 muteins with improved tolerability and IL-2-mediated NK and T cell effector functions would allow for administration of similar therapeutic doses that are better tolerated or higher therapeutic doses, thereby increasing the potential for greater therapeutic efficacy of this protein. The overall strategy of the work presented herein was to select novel human IL-2 muteins that exhibit the following functional profile using a comprehensive panel of specialized moderate throughput human NK cell-based immunoassay screening systems: reduced pro-inflammatory cytokine production (particularly TNF-α) so as to improve tolerability, and improved NK cell-mediated function as reflected in the ability of the mutein to maintain or increase NK and/or T cell proliferation, to maintain or increase NK-mediated cytotoxicity (NK, LAK, and ADCC), and to maintain or increase NK cell survival.

For purposes of identifying suitable IL-2 muteins with the desired therapeutic profile, the biological activities of the candidate recombinant human IL-2 muteins were compared to these biological activities exhibited by des-alanyl-1, C125S human IL-2 (abbreviated as “Pro” in the examples below) and C125S human IL-2 (abbreviated as “Ala-Pro” in the examples below), which are referred to as the reference IL-2 muteins. The recombinantly E. coli-produced des-alanyl-1, C125S human IL-2 mutein, which is aldesleukin, is marketed as a formulation under the tradename Proleukin® IL-2 (Chiron Corporation, Emeryville, Calif.). Proleukin® IL-2 is a specific lyophilized formulation that uses an unglycosylated form of the mutein that has been produced in E. coli, and was reconstituted in distilled water for use in the bioassays described herein below. The AME mammalian expression systems DirectAME™ and ExpressAME™ (Applied Molecular Evolution, Inc., San Diego, Calif.) were utilized in the recombinant production of the C125S human IL-2 used in the initial screening experiments.

The human IL-muteins described herein below were expressed in host mammalian 293T cells. Where the reference IL-2 mutein was C125S human IL-2, the host cells had been transformed with an expression contruct comprising the native human IL-2 coding sequence with a C125S mutation operably linked to the Pro-1 promoter. The coding sequence comprised the authentic IL-2 signal sequence and codon for the N-terminal alanine of human IL-2 (i.e., nucleotides 1-63 of SEQ ID NO:1) fused at the coding sequence for des-alanyl-1, C125S human IL-2 (i.e., SEQ ID NO:7). The protein was expressed as GSHis-tagged protein in the 293T cell mammalian expression system and purified with NI-NTA beads.

### Example 1

### Initial Screening of Human IL-2 Muteins

A library comprising all 2,508 possible single amino acid mutein variants of the C125S human IL-2 molecule (designated “Ala-Pro” in the examples herein) was constructed using a codon-based mutagenesis technology platform (Applied Molecular Evolution, Inc., San Diego, Calif.). Ala-Pro differs from the des-alanyl-1, C125S human IL-2 mutein utilized in the commercially available Proleukin® IL-2 product in having the N-terminal Ala residue at position 1 of the naturally occurring mature human IL-2 sequence retained in the C125S human IL-2 mutein. The AME mammalian expression systems DirectAME™ and ExpressAME™ (Applied Molecular Evolution, Inc., San Diego, Calif.) were utilized in the recombinant production of the Ala-Pro muteins.

The primary screen was carried out using a human NK-92 cell line-based functional immunoassay, which assayed pro-inflammatory cytokine production (TNF-α) and NK cell proliferation, and NK cytolytic killing (NK, LAK, and ADCC) and cell survival (PAKT) were assayed using the human NK3.3 cell line. The primary functional endpoints selected included: (1) reduced pro-inflammatory TNF-α production by the human NK-92 cell line relative to that observed with Ala-Pro IL-2 (i.e., C125S human IL-2 mutein) or Proleukin®IL-2 (i.e., des-alanyl-1, C125S human IL-2 mutein); (2) maintained or improved human NK-92 cell line proliferation relative to that observed with either of these two reference IL-2 muteins; and 3) maintained or improved human NK3.3 cell line-mediated NK-, LAK-, and ADCC-mediated cytolytic killing relative to that observed with either of these two reference IL-2 muteins. Secondary functional endpoints were maintained or improved induction of phosphorylated AKT (PAKT) in the NK3.3 cell line relative to that observed with either of these two reference IL-2 muteins and maintained or improved T cell proliferation by the human Kit225 T cell line relative to that observed with Ala-Pro IL-2 (i.e., C25S human IL-2 mutein) or Proleukin®IL-2 (i.e., comprising the des-alanyl-1, C125S human IL-2 mutein).

The initial screening process identified 168 single-amino-acid substitutions (see copending U.S. Patent Application No. 60/550,868, entitled “Improved Interleukin-2 Muteins,” Attorney Docket No. PP20354.001 (035784/261164), filed Mar. 5, 2004; herein incorporated by reference in its entirety) within the C125S human IL-2 mutein that were then combined in three combinatorial libraries designed to combine the desirable functional profiles of these 168 single-amino-acid variants of the C125S human IL-2 mutein to find additional IL-2 muteins with increased tolerability (i.e., reduced IL-2 induction of TNF-α production by NK cells) and maintained or increased NK cell effector function. The combinatorial libraries were comprised of 753 IL-2 muteins with multiple amino acid substitutions ranging from a minimum of one substitution to as many as six possible amino acid substitutions (i.e., in addition to the C125S substitution of the naturally occurring mature human IL-2 sequence).

Out of these three combinatorial libraries, 32 combinatorial muteins (see Table 1 herein above for combination substitutions in these muteins) having the desired functional profile (as compared to des-alanyl-1, C125S human IL-2 or C125S human IL-2) were identified using a comprehensive panel of specialized human NK and T cell-based moderate throughput immunoassay systems that were developed to quantitate IL-2-dependent human NK- and T-cell-line proliferation, pro-inflammatory cytokine production (TNF-α) by NK cells, and NK-mediated cytolytic activity (NK/LAK/ADCC). The screening data for all 32 muteins is shown in Table 7 herein below.

Subsequent analysis following screening of these muteins in extended dose response ranges resulted in identification of specific muteins comprising three distinct functional classes predictive of improved clinical benefit, described in Examples 2-4 below. All IL-2 muteins selected maintain NK cytolytic function (NK/LAK/ADCC) when compared to the des-alanyl-1, C125S or C125S human IL-2 muteins. The following protocols were used in the screening process.

NK Cell Proliferation/TNF-α Production

The IL-2 bioassay for natural killer (NK) cell proliferation and TNF-α production utilizes the human NK-92 cell line (ATCC CRL-2407, CMCC ID #11925). The NK-92 cell line, originally described by Gong et al. (1994) Leukemia 8(4):652-658, displays phenotypic and functional characteristics of activated NK cells. Proliferation of NK-92 is IL-2 dependent; cells will die if cultured in the absence of IL-2 for 72 hours. The cell line also produces detectable levels of TNF-α within 48-72 hours following exposure to IL-2.

NK-92 cells were cultured in complete medium (NK-92 medium) consisting of Alpha-MEM, 12% heat-inactivated fetal bovine serum (FBS), 8% heat-inactivated horse serum, 0.02 mM folic acid, 0.2 mM inositol, 2 mM L-glutamine, and 0.1 mM β-mercaptoethanol. Cultures were seeded at a minimum density of 1-3×10⁵ cells/ml and supplemented with 1000 IU/ml recombinant human IL-2 mutein (des-alanyl-1, C125S human IL-2 (i.e., aldesleukin or Proleukin® IL-2; Chiron Corporation, Emeryville, Calif.) or C125S human IL-2 (recombinantly produced in the AME's mammalian expression system noted above). In preparation for the assay, cells were placed in fresh NK-92 medium a minimum of 48 h prior to assay use. One day prior to assay, NK-92 were washed three times and placed in NK-92 medium without any supplemental IL-2 for 24 h. Cells were centrifuged, suspended in NK-92 medium (no IL-2) and plated into 96-well flat bottom plates at a density of 4×10⁴ cells/well in 200 μl with varying concentrations of des-alanyl-1, C125S or C125S human IL-2 as the reference IL-2 molecule or varying concentrations of an IL-2 mutein of the invention diluted in NK-92 medium. Following a 72-h incubation at 37° C., 5% CO₂, a 100 μl aliquot of culture supernatant was removed and frozen for subsequent quantification of TNF-α using a commercially available TNF-α ELISA kit (BioSource Cytoscreen™ Human TNF-α ELISA kit; Camarillo, Calif.). For the remaining cells in culture, proliferation was determined using a commercially available MTT dye-reduction kit (CellTiter 96® Non-Radioactive Cell Proliferation Assay Kit (Promega Corp., Madison, Wis.), and a stimulation index was then calculated based on a colorimetric readout.

NK Cell-Mediated Cytotoxicity

The IL-2 bioassay for natural killer (NK) cell-mediated cytotoxicity utilizes the human NK3.3 cell line. The NK3.3 cell line displays phenotypic and functional characteristics of peripheral blood NK cells (Kombluth (1982) J. Immunol. 129(6):2831-2837), and can mediate antibody-dependent cellular cytotoxicity (ADCC) via the Fc receptor (CD16, FcγRIII). The cell line was obtained from Jackie Kombluth, Ph.D., under limited use license agreement with St. Louis University, and deposited to CMCC (ID 12022).

Table 2 summarizes the biological activities of NK3.3 cells examined with this IL-2 bioassay.

TABLE 2
Biological activities of NK3.3 cells examined with IL-2 bioassay.

| ACTIVITY | EFFECTOR | TARGET | DESCRIPTION |
|---|---|---|---|
| NK | NK3.3 | K562 | Natural cytotoxicity |
| LAK | NK3.3 | Daudi | IL-2 activated killing |
| ADCC | NK3.3 | Daudi + Rituxan ® | Antibody-dependent |
| | | | cellular cytotoxicity |

NK3.3 cells were expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for the assay, NK3.3 cells were cultured in the absence of IL-2 (“starved”) for 24 h. The assay consists of 5×10⁴ “starved” NK3.3 cells plated in U-bottom 96-well plates, exposed to varying concentrations of des-alanyl-1, C125S or C125S human IL-2 as the reference IL-2 molecule or varying concentrations of an IL-2 mutein of the invention in a total volume of 100 μl. Following an 18-h incubation, the IL-2-stimulated NK3.3 effector cells were co-incubated with 5×10³ calcein AM-labeled target cells (K562 or Daudi) or antibody-coated, calcein AM-labeled targets (Daudi coated with rituximab at a final concentration of 2 μg/ml) to achieve a final effector-to-target ratio of 10:1 in final volume of 200 μl. Following co-incubation of effector and target cells for 4 h, the 96 well plates were briefly centrifuged; 100 μl of culture supernatant was removed and placed into a black, clear-bottom, flat-bottom 96-well plate for quantitation of calcein AM release by fluorimeter. Quantitation was expressed as percent specific lysis, and was calculated by the following equation: % specific lysis=100×[(mean experimental−mean spontaneous release)/(mean maximal release−mean spontaneous release)]; whereby the spontaneous release was determined from wells containing labeled targets and no effectors, and maximal release was determined from wells containing labeled targets and 1% Triton X-100.

T-Cell Proliferation

The IL-2 bioassay for T-cell proliferation utilizes the human T-cell line Kit225 (CMCC ID#1 1234), derived from a patient with T-cell chronic lymphocytic leukemia (Hori et al. (1987) Blood 70(4): 1069-1072). Kit 225 cells constitutively express the α, β, γ subunits of the IL-2 receptor complex. Proliferation of Kit225 is IL-2 dependent; cells will die if cultured in the absence of IL-2 for an extended period of time. The assay consists of Kit225 cells, cultured in the absence of IL-2 for 24 h, followed by plating a specified number of cells with varying concentrations of des-alanyl-1, C125S or C125S human IL-2 as the reference IL-2 molecule or varying concentrations of an IL-2 mutein of the invention. Following a 48-h incubation, proliferation was determined using a standard, commercially available MTT dye reduction kit, and a stimulation index was calculated based on a colorimetric readout.

NK Cell Survival Signaling

A subset of the human IL-2 mutein library was screened for the ability to induce NK cell survival signaling. Proleukin® IL-2 (i.e., formulation comprising aldesleukin, the des-alanyl-1, C125S human IL-2 mutein) induces the phosphorylation of AKT in NK3.3 cells previously starved for IL-2, which is considered a “survival signal.” NK3.3 cells were expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for assay, NK3.3 cells were cultured in the absence of IL-2 for 24 h. As an indicator of cell survival signaling, “starved” NK3.3 cells (2×10⁶) were stimulated by addition of 2 nM of des-alanyl-1, C125S or C125S human IL-2 as the reference IL-2 molecule or 2 nM of an IL-2 mutein of the invention, for 30 min. Cells were washed twice in phosphate buffered saline (PBS). The cell pellet was lysed in 50 μl of a cell extraction buffer containing protease inhibitors and subjected to one freeze-thaw cycle. The extract was centrifuged at 13,000 rpm for 10 min at 4° C. An aliquot of the cleared lysate was added at a 1:10 dilution to wells of the AKT [pS473]*Imunoassay Kit (BioSource International). Following the manufacturer's protocol, levels of phosphorylated AKT were detected by quantitative ELISA.

### Example 2

### Identification of Beneficial Mutations that Reduce TNF-α Production by NK Cells

The first functional class of muteins is predicted to have improved tolerability as evidenced by an impaired induction of TNF-α production by NK cells that is <60% of that observed with the C125S human IL-2 mutein (designated “Ala-Pro” in the data herein below) when assayed at 1.0 nM. The muteins within this class fall within two categories:
(1) those that induce low TNF-α production by NK cells and maintain NK cell proliferation at a mutein concentration of 1.0 nM (i.e., 1000 pM), but proliferative activity drops at lower concentrations of the mutein, which include the des-alanyl-1, C125S or C125S human IL-2 muteins further comprising the 19D40D, 36D61R, 36D65L, 40D61R, 40D65Y, 40G65Y, or 81K91D combination substitution, where the residue position (i.e., 19, 36, 40, 61, 65, 81, or 91) is relative to the mature human IL-2 sequence (i.e., relative to SEQ ID NO:4), which are shown in Table 3 below; and

(2) those that induce low TNF-α production by NK cells, and where proliferative activity is maintained down to 50 pM; furthermore, the TNF-α production by NK cells must have been <80% that of C125S human IL-2 at 0.05 nM (i.e., 50 pM) and 0.1 nM (i.e., 100 pM); this subclass includes the des-alanyl-1, C125S or C125S human IL-2 muteins further comprising the 40D72N, 80K65Y, 81K88D, 81K42E, 81K72N, 107H65Y, or 107R72N combination substitution, where the residue position (i.e., 40, 42, 65, 72, 80, 81, 88, or 107) is relative to the mature human IL-2 sequence (i.e., relative to SEQ ID NO:4), which are shown in Table 4 below.

TABLE 3
IL-2 muteins identified as having reduced induction of TNF-α production by NK cells. TNF-α production
by NK cells at the various concentrations of IL-2 mutein is expressed as a ratio of that observed for
C125S human IL-2 (:Ala-Pro). NK cell proliferation (NK92-MTT) at the various concentrations of IL-2
mutein is expressed as a ratio of that observed for C125S human IL-2 (:Ala-Pro).

| | TNF-α | | | | NK92 MTT | | | |
|---|---|---|---|---|---|---|---|---|
| | :Ala-Pro | :Ala-Pro | :Ala-Pro | :Ala-Pro | :Ala-Pro | :Ala-Pro | :Ala-Pro | :Ala-Pro |
| Sequence | 20 pM | 50 pM | 100 pM | 1000 pM | 20 pM | 50 pM | 100 pM | 1000 pM |
| 19D40D | 0.56 | 0.22 | 0.21 | 0.37 | 0.61 | 0.38 | 0.24 | 1.17 |
| 36D61R | 0.37 | 0.44 | 0.40 | 0.47 | 0.38 | 0.64 | 0.83 | 1.27 |
| 36D65L | 0.56 | 0.52 | 0.45 | 0.52 | 0.38 | 0.73 | 1.01 | 1.20 |
| 40D61R | 0.06 | 0.30 | 0.23 | 0.26 | 0.33 | 0.57 | 0.95 | 1.33 |
| 40D65Y | 0.24 | 0.33 | 0.21 | 0.28 | 0.31 | 0.50 | 0.58 | 1.35 |
| 40G65Y | 0.33 | 0.25 | 0.34 | 0.33 | 0.30 | 0.22 | 0.42 | 1.21 |
| 81K91D | 0.42 | 0.14 | 0.29 | 0.26 | 0.41 | 0.58 | 0.90 | 1.29 |

TABLE 4
Additional IL-2 muteins identified as having reduced induction of TNF-α production
by NK cells. TNF-α production by NK cells at the various concentrations of IL-2
mutein is expressed as a ratio of that observed for C125S human IL-2 (:Ala-Pro).
NK cell proliferation (NK92-MTT) at the various concentrations of IL-2 mutein is
expressed as a ratio of that observed for C125S human IL-2 (:Ala-Pro).

| | TNF-α | | | | NK92 MTT | | | |
|---|---|---|---|---|---|---|---|---|
| | :Ala-Pro | :Ala-Pro | :Ala-Pro | :Ala-Pro | :Ala-Pro | :Ala-Pro | :Ala-Pro | :Ala-Pro |
| Sequence | 20 pM | 50 pM | 100 pM | 1000 pM | 20 pM | 50 pM | 100 pM | 1000 pM |
| 40D72N | 0.02 | 0.44 | 0.45 | 0.35 | 0.46 | 0.92 | 1.04 | 1.30 |
| 80K65Y | 0.47 | 0.60 | 0.55 | 0.48 | 0.67 | 1.16 | 1.37 | 1.36 |
| 81K88D | 0.37 | 0.58 | 0.49 | 0.59 | 0.63 | 1.10 | 1.12 | 1.25 |
| 81K42E | 0.19 | 0.63 | 0.55 | 0.55 | 0.60 | 1.30 | 1.35 | 1.16 |
| 81K72N | 0.60 | 0.80 | 0.73 | 0.59 | 0.92 | 1.19 | 1.50 | 1.07 |
| 107H65Y | 0.70 | 0.74 | 0.55 | 0.55 | 0.74 | 1.03 | 1.12 | 1.16 |
| 107R72N | 0.93 | 0.74 | 0.65 | 0.48 | 0.87 | 1.30 | 1.43 | 1.00 |

### Example 3

### Identification of Beneficial Mutations that Enhance NK Cell Proliferation

The second functional class of human IL-2 muteins enhances NK cell proliferation >200% compared to C125S human IL-2 at one or more concentrations tested (5 pM, 20 pM, 50 pM, 100 pM, and 1000 pM) without deleterious impact on TNF-α production (<100% TNF-α production relative to that observed for the reference IL-2 mutein at a concentration of 100 pM or 1 nM). Furthermore, selection criteria included a proliferation index greater than 150% of that observed for the reference IL-2 mutein, i.e., C125S human IL-2 (Ala-Pro) for at least 2 concentrations tested. This functional class includes the des-alanyl-1, C125S or C125S human IL-2 muteins further comprising the 19D81K, 40G36D, or 81K36D substitution, where the residue position (i.e., 19, 36, 40, or 81) is relative to the mature human IL-2 sequence (i.e., relative to SEQ ID NO:4). See Table 5 below.

TABLE 5
IL-2 muteins identified as having enhanced induction of NK cell proliferation without negatively impacting TNF-α production
by NK cells. TNF-α production by NK cells at the various concentrations of IL-2 mutein is expressed as a ratio
of that observed for C125S human IL-2 (:Ala-Pro). NK cell proliferation (NK92-MTT) at the various concentrations
of IL-2 mutein is expressed as a ratio of that observed for C125S human IL-2 (:Ala-Pro).

| | TNF-α | | | | | NK92 MTT | | | | |
|---|---|---|---|---|---|---|---|---|---|---|
| | :Ala-Pro | :Ala-Pro | :Ala-Pro | :Ala-Pro | :Ala-Pro | :Ala-Pro | :Ala-Pro | :Ala-Pro | :Ala-Pro | :Ala-Pro |
| Sequence | 5 pM | 20 pM | 50 pM | 100 pM | 1000 pM | 5 pM | 20 pM | 50 pM | 100 pM | 1000 pM |
| 19D81K | 0.91 | 1.12 | 0.97 | 0.76 | 0.75 | 0.65 | 1.47 | 1.69 | 1.56 | 1.05 |
| 40G36D | 0.97 | 1.31 | 1.09 | 0.95 | 0.73 | 1.22 | 2.16 | 2.94 | 2.36 | 1.04 |
| 81K36D | 0.54 | 0.42 | 1.00 | 1.06 | 0.85 | 1.01 | 1.62 | 1.81 | 2.19 | 1.04 |

### Example 4

### Identification of “Bi-Functional” Mutations

The third functional class of human IL-2 muteins shows increased proliferative activity and decreased TNF-α production by NK cells, where TNF-α production is <75% of that observed for the C125S human IL-2 mutein when tested at 1 nM, and proliferation of NK cells is >150% of that observed for the C125S human IL-2 mutein at any one concentration tested (5 pM, 20 pM, 50 pM, 100 pM, and 1000 pM). This group includes the des-alanyl-1, C125S human IL-2 mutein or the C125S human IL-2 mutein further comprising the 36D42R, 36D80K, 40D80K, 81K61R, 91N95G, 107H36D, 107R36D, or 91N94Y95G substitution, where the residue position (i.e., 36, 40, 42, 61, 80, 81, 91, 94, 95, or 107) is relative to the mature human IL-2 sequence (i.e., relative to SEQ ID NO:4). See Table 6 below.

TABLE 6
Bi-functional IL-2 muteins identified as having enhanced induction of NK cell proliferation
and decreased induction of TNF-α production by NK cells. TNF-α production by NK cells
at the various concentrations of IL-2 mutein is expressed as a ratio of that observed for C125S
human IL-2 (:Ala-Pro). NK cell proliferation (NK92-MTT) at the various concentrations of IL-2
mutein is expressed as a ratio of that observed for C125S human IL-2 (:Ala-Pro).

| | TNF-α | | | | NK92 MTT | | | |
|---|---|---|---|---|---|---|---|---|
| | :Ala-Pro | :Ala-Pro | :Ala-Pro | :Ala-Pro | :Ala-Pro | :Ala-Pro | :Ala-Pro | :Ala-Pro |
| Sequence | 20 pM | 50 pM | 100 pM | 1000 pM | 20 pM | 50 pM | 100 pM | 1000 pM |
| 40D80K | 0.60 | 0.86 | 0.65 | 0.64 | 1.52 | 1.93 | 1.58 | 1.07 |
| 91N95G | 0.84 | 0.89 | 0.79 | 0.57 | 1.35 | 1.71 | 1.89 | 1.12 |
| 107H36D | 0.70 | 1.06 | 0.76 | 0.69 | 1.41 | 2.36 | 2.13 | 1.08 |
| 107R36D | 0.47 | 0.77 | 0.68 | 0.67 | 0.74 | 1.60 | 1.83 | 1.14 |
| 81K61R | 0.19 | 0.91 | 0.80 | 0.61 | 1.04 | 2.06 | 1.82 | 1.14 |
| 36D42R | 0.28 | 0.55 | 0.77 | 0.60 | 1.30 | 1.26 | 1.97 | 1.04 |
| 80K36D | 0.51 | 0.60 | 0.53 | 0.50 | 0.80 | 1.52 | 1.43 | 1.24 |
| 91N94Y95G | 0.88 | 0.80 | 0.67 | 0.41 | 1.25 | 1.82 | 1.60 | 1.34 |

Table 7 below summarizes the functional profile of the 32 combinatorial muteins identified in this screening process.

TABLE 7
Functional profile for the 32 combinatorial muteins identified in the screening process. TNF-α production
by NK cells at the various concentrations of IL-2 mutein is shown. Total TNF-α production by NK cells
at the various concentrations of IL-2 mutein is also expressed as a ratio of that observed for C125S human
IL-2 (:Ala-Pro). NK cell proliferation (NK92-MTT), Kit225, and NK activity at the various concentrations
of IL-2 mutein are expressed as a ratio of that observed for C125S human IL-2 (:Ala-Pro).

| | TNF-α (pg/ml) | | | | | TNF-α :Ala-Pro | | | | |
|---|---|---|---|---|---|---|---|---|---|---|
| Sequence | 5 pM | 20 pM | 50 pM | 100 pM | 1000 pM | 5 pM | 20 pM | 50 pM | 100 pM | 1000 pM |
| 19D40D | 7.6 | 7.2 | 5.2 | 8.3 | 67.0 | 1.03 | 0.56 | 0.22 | 0.21 | 0.37 |
| 19D81K | 6.7 | 14.4 | 23.1 | 29.7 | 135.2 | 0.91 | 1.12 | 0.97 | 0.76 | 0.75 |
| 36D42R | 5.8 | 3.7 | 13.0 | 30.3 | 108.4 | 0.78 | 0.28 | 0.55 | 0.77 | 0.60 |
| 36D61R | 4.9 | 4.8 | 10.4 | 15.8 | 85.4 | 0.66 | 0.37 | 0.44 | 0.40 | 0.47 |
| 36D65L | 4.9 | 7.2 | 12.4 | 17.5 | 95.0 | 0.66 | 0.56 | 0.52 | 0.45 | 0.52 |
| 40D36D | 5.3 | 16.3 | 32.7 | 40.2 | 164.4 | 0.72 | 1.26 | 1.37 | 1.03 | 0.91 |
| 40D61R | 7.1 | 0.8 | 7.1 | 8.9 | 47.9 | 0.97 | 0.06 | 0.30 | 0.23 | 0.26 |
| 40D65Y | 8.0 | 3.1 | 7.8 | 8.3 | 50.8 | 1.09 | 0.24 | 0.33 | 0.21 | 0.28 |
| 40D72N | 5.3 | 0.2 | 10.4 | 17.5 | 63.3 | 0.72 | 0.02 | 0.44 | 0.45 | 0.35 |
| 40D80K | 2.2 | 7.8 | 20.4 | 25.6 | 115.8 | 0.30 | 0.60 | 0.86 | 0.65 | 0.64 |
| 40G36D | 7.1 | 16.9 | 25.8 | 37.3 | 131.5 | 0.97 | 1.31 | 1.09 | 0.95 | 0.73 |
| 40G65Y | 4.4 | 4.2 | 5.8 | 13.5 | 60.3 | 0.60 | 0.33 | 0.25 | 0.34 | 0.33 |
| 80K36D | 5.3 | 6.6 | 14.4 | 21.0 | 89.8 | 0.72 | 0.51 | 0.60 | 0.53 | 0.50 |
| 80K65Y | 4.4 | 6.0 | 14.4 | 21.5 | 87.6 | 0.60 | 0.47 | 0.60 | 0.55 | 0.48 |
| 81K107H | 7.1 | 75.7 | 34.1 | 37.3 | 130.0 | 0.97 | 5.86 | 1.43 | 0.95 | 0.72 |
| 81K36D | 4.0 | 5.4 | 23.8 | 41.4 | 153.9 | 0.54 | 0.42 | 1.00 | 1.06 | 0.85 |
| 81K42E | 7.1 | 2.5 | 15.0 | 21.5 | 100.2 | 0.97 | 0.19 | 0.63 | 0.55 | 0.55 |
| 81K61R | 3.5 | 2.5 | 21.8 | 31.4 | 111.4 | 0.48 | 0.19 | 0.91 | 0.80 | 0.61 |
| 81K65Y | 4.0 | 9.0 | 23.1 | 33.8 | 134.5 | 0.54 | 0.70 | 0.97 | 0.86 | 0.74 |
| 81K72N | 4.4 | 7.8 | 19.1 | 28.5 | 106.2 | 0.60 | 0.60 | 0.80 | 0.73 | 0.59 |
| 81K88D | 3.1 | 4.8 | 13.7 | 19.2 | 107.6 | 0.42 | 0.37 | 0.58 | 0.49 | 0.59 |
| 81K91D | 6.7 | 5.4 | 3.3 | 11.2 | 46.4 | 0.91 | 0.42 | 0.14 | 0.29 | 0.26 |
| 81L107H | 9.0 | 34.9 | 37.5 | 43.2 | 191.4 | 1.22 | 2.70 | 1.58 | 1.10 | 1.06 |
| 91N95G | 5.8 | 10.8 | 21.1 | 30.9 | 103.2 | 0.78 | 0.84 | 0.89 | 0.79 | 0.57 |
| 107H36D | 5.8 | 9.0 | 25.2 | 29.7 | 124.8 | 0.78 | 0.70 | 1.06 | 0.76 | 0.69 |
| 107H42E | 6.2 | 7.2 | 12.4 | 11.2 | 83.2 | 0.85 | 0.56 | 0.52 | 0.29 | 0.46 |
| 107H65Y | 5.8 | 9.0 | 17.7 | 21.5 | 100.2 | 0.78 | 0.70 | 0.74 | 0.55 | 0.55 |
| 107R36D | 3.1 | 6.0 | 18.4 | 26.8 | 121.8 | 0.42 | 0.47 | 0.77 | 0.68 | 0.67 |
| 107R72N | 7.6 | 12.0 | 17.7 | 25.6 | 86.9 | 1.03 | 0.93 | 0.74 | 0.65 | 0.48 |
| 40D81K107H | 9.4 | 13.2 | 28.6 | 32.6 | 118.1 | 1.28 | 1.02 | 1.20 | 0.83 | 0.65 |
| 40G81K107H | 5.3 | 7.8 | 19.7 | 27.9 | 126.3 | 0.72 | 0.60 | 0.83 | 0.71 | 0.70 |
| 91N94Y95G | 6.2 | 11.4 | 19.1 | 26.2 | 74.3 | 0.85 | 0.88 | 0.80 | 0.67 | 0.41 |

| | NK92 MTT :Ala-Pro | | | | | KIT225 MTT | | | | | NK |
|---|---|---|---|---|---|---|---|---|---|---|---|
| | 5 | 20 | 50 | 100 | 1000 | 5 | 20 | 50 | 200 | 500 | 100 |
| Sequence | pM | pM | pM | pM | pM | pM | pM | pM | pM | pM | pM |
| 19D40D | 0.61 | 0.38 | 0.24 | 0.33 | 1.17 | 0.82 | 0.80 | 0.74 | 0.86 | 1.09 | 0.78 |
| 19D81K | 0.65 | 1.47 | 1.69 | 1.56 | 1.05 | 0.86 | 0.95 | 1.08 | 1.14 | 1.10 | 0.78 |
| 36D42R | 1.83 | 1.30 | 1.26 | 1.97 | 1.04 | 0.83 | 0.74 | 0.65 | 0.70 | 1.04 | 1.12 |
| 36D61R | 0.93 | 0.38 | 0.64 | 0.83 | 1.27 | 0.83 | 0.73 | 0.63 | 0.67 | 0.99 | 0.78 |
| 36D65L | 1.05 | 0.38 | 0.73 | 1.01 | 1.20 | 0.80 | 0.73 | 0.64 | 0.68 | 1.00 | 0.86 |
| 40D36D | 1.50 | 2.45 | 3.23 | 2.14 | 1.01 | 0.95 | 1.02 | 1.15 | 1.10 | 1.15 | 0.99 |
| 40D61R | 0.65 | 0.33 | 0.57 | 0.95 | 1.33 | 0.84 | 0.79 | 0.67 | 0.76 | 1.11 | 0.91 |
| 40D65Y | 0.67 | 0.31 | 0.50 | 0.58 | 1.35 | 0.85 | 0.79 | 0.75 | 0.83 | 1.07 | 0.69 |
| 40D72N | 0.75 | 0.46 | 0.92 | 1.04 | 1.30 | 0.84 | 0.85 | 0.81 | 0.95 | 1.04 | 0.71 |
| 40D80K | 0.74 | 1.52 | 1.93 | 1.58 | 1.07 | 0.88 | 1.02 | 1.11 | 1.15 | 1.14 | 0.91 |
| 40G36D | 1.22 | 2.16 | 2.94 | 2.36 | 1.04 | 0.95 | 1.12 | 1.10 | 1.20 | 1.12 | 1.18 |
| 40G65Y | 0.72 | 0.30 | 0.22 | 0.42 | 1.21 | 0.81 | 0.76 | 0.74 | 0.82 | 0.77 | 0.76 |
| 80K36D | 0.92 | 0.80 | 1.52 | 1.43 | 1.24 | 0.90 | 0.93 | 1.00 | 1.11 | 1.10 | 1.00 |
| 80K65Y | 0.77 | 0.67 | 1.16 | 1.37 | 1.36 | 0.88 | 0.92 | 1.01 | 1.09 | 1.08 | 0.88 |
| 81K107H | 1.27 | 1.92 | 2.49 | 2.06 | 1.07 | 1.05 | 1.18 | 1.15 | 1.19 | 1.10 | 0.92 |
| 81K36D | 1.01 | 1.62 | 1.81 | 2.19 | 1.04 | 0.94 | 1.06 | 1.09 | 1.16 | 1.13 | 0.87 |
| 81K42E | 0.83 | 0.60 | 1.30 | 1.35 | 1.16 | 0.84 | 0.84 | 0.85 | 0.91 | 1.07 | 0.96 |
| 81K61R | 0.72 | 1.04 | 2.06 | 1.82 | 1.14 | 0.88 | 0.98 | 1.06 | 1.15 | 1.12 | 1.00 |
| 81K65Y | 0.89 | 1.19 | 1.27 | 1.40 | 1.03 | 0.91 | 1.02 | 1.01 | 1.08 | 1.11 | 1.02 |
| 81K72N | 0.87 | 0.92 | 1.19 | 1.50 | 1.07 | 0.92 | 1.01 | 1.04 | 1.08 | 1.09 | 1.10 |
| 81K88D | 0.61 | 0.63 | 1.10 | 1.12 | 1.25 | 0.85 | 0.86 | 0.97 | 1.05 | 1.12 | 0.86 |
| 81K91D | 0.80 | 0.41 | 0.58 | 0.90 | 1.29 | 0.88 | 0.91 | 0.96 | 1.09 | 1.16 | 0.89 |
| 81L107H | 1.14 | 1.97 | 2.35 | 1.90 | 0.97 | 0.96 | 1.02 | 1.14 | 1.11 | 1.12 | 0.89 |
| 91N95G | 1.08 | 1.35 | 1.71 | 1.89 | 1.12 | 0.90 | 1.02 | 1.07 | 1.10 | 0.96 | 1.13 |
| 107H36D | 0.99 | 1.41 | 2.36 | 2.13 | 1.08 | 0.88 | 0.99 | 1.06 | 1.12 | 1.05 | 1.14 |
| 107H42E | 0.68 | 0.50 | 0.59 | 0.58 | 1.19 | 0.85 | 0.87 | 0.83 | 0.89 | 0.99 | 0.93 |
| 107H65Y | 0.72 | 0.74 | 1.03 | 1.12 | 1.16 | 0.90 | 0.92 | 0.98 | 1.03 | 1.10 | 1.00 |
| 107R36D | 0.92 | 0.74 | 1.60 | 1.83 | 1.14 | 0.84 | 0.78 | 0.76 | 0.88 | 1.04 | 0.79 |
| 107R72N | 0.69 | 0.87 | 1.30 | 1.43 | 1.00 | 0.89 | 0.97 | 1.00 | 1.08 | 1.03 | 1.04 |
| 40D81K107H | 0.80 | 1.72 | 2.56 | 1.83 | 1.01 | 0.95 | 1.08 | 1.14 | 1.18 | 1.08 | 0.92 |
| 40G81K107H | 0.67 | 0.90 | 1.23 | 1.55 | 1.01 | 0.92 | 1.04 | 1.08 | 1.12 | 1.15 | 0.71 |
| 91N94Y95G | 0.91 | 1.25 | 1.82 | 1.60 | 1.34 | 0.94 | 1.07 | 1.07 | 1.13 | 1.10 | 0.79 |

### Example 5

### Identification of Beneficial IL-2 Mutations that Reduce Pro-inflammatory Cytokine Production while Maintaining or Increasing Levels of Proliferation and Cytotoxicity in Normal Human Peripheral Blood Mononuclear Cells

From the combinatorial amino acid substitution series of 32 IL-2 muteins described above, 18 IL-2 muteins were selected for a small-scale expression/purification as indicated in Table 8. These IL-2 muteins were tested for their ability to generate a similar functional profile of increased tolerability and maintained activity in peripheral blood mononuclear cells (PBMC) isolated from several normal human blood donors, as compared to relevant IL-2 controls (des-alanyl-1, C125S human IL-2 mutein (present in Proleukin®) and yeast-expressed des-alanyl-1, C125S human IL-2 mutein (designated Y-Pro in the data described below). Specifically, purified IL-2 muteins were screened by stimulating human PBMC derived from a panel of normal human donors, and assaying for proliferation and pro-inflammatory cytokine production (TNF-α), as well as the ability to kill tumor cell targets by natural/spontaneous cytotoxicity (NK), lymphokine-activated killing (LAK), or antibody dependent cellular cytotoxicity (ADCC).

TABLE 8
Human IL-2 muteins comprising the amino acid sequence of
C125S human IL-2 (SEQ ID NO: 6) or des-alanyl-1, C125S human
IL-2 (SEQ ID NO: 8) with the following combination substitutions
were screened for activity in human PBMC.¹

| | | | | |
|---|---|---|---|---|
| 19D40D | 80K65Y | 91N95G | 40D72N | 81K61R |
| 40G36D | 81K91D | 40D65Y | 81K42E | 107R72N |
| 81K88D | 40D61R | 81K36D | 107R36D | |
| 36D42R | 80K36D | 107H36D | 40D80K | |

¹IL-2 muteins identified by: amino acid position relative to mature human IL-2 of SEQ ID NO: 4, and amino acid substitution at that position.

The following primary functional endpoints were used:
1) Reduced pro-inflammatory cytokine production (TNF-α) by human PBMC stimulated with IL-2 mutein as compared to relevant human IL-2 mutein control;
2) Maintained or improved IL-2 induced proliferation in human PBMC without an increase in pro-inflammatory cytokine production as compared to relevant human IL-2 mutein control; and
3) Maintained or improved NK, LAK, and ADCC mediated cytolytic killing by human PBMC stimulated in vitro with IL-2 mutein as compared to relevant human IL-2 mutein control.
   Assay Descriptions
   Combination Proliferation/Proinflammatory Cytokine Production Assay Procedure

Upon exposure to IL-2, human PBMC proliferate and secrete cytokines in a dose-dependent manner. To maximize data output and efficiency, a combination assay was designed to assess levels of proliferation and cytokine production following 72 h stimulation with the reference IL-2 mutein or the human IL-2 mutein of interest. The assay setup involves isolation of PBMC by density gradient separation (ACDA Vacutainer CPT tubes) from one or more normal human donors. In 96-well tissue-culture treated plates, 200,000 cells per well are incubated with various concentrations of IL-2 (0.039 nM-10 nM) or no IL-2 as a negative control in complete RPMI medium (RPMI, 10% heat-inactivated human AB serum, 25 mM HEPES, 2 mM glutamine, penicillin/streptomycin/fungizone) at 37° C., 7% CO₂. Following 66 h of incubation, an aliquot of cell culture supernatant is removed and frozen for cytokine detection at a later time. The cells are pulsed with 1 μCi ³H-thymidine for 6 h then harvested to determine levels of nucleotide incorporation (Wallac Trilux Microbeta Plate Reader) as a measure of cell proliferation. Commercially available ELISA kits (BioSource International) were used to detect levels of TNF-α in the cell culture supernatants per manufacturer's guidelines. Repeating the assay for a complete panel of six separate donors provides a characterization of representative proliferative and cytokine responses to IL-2 in a “normal population.”

Data Analysis

PBMC samples were plated in duplicate in separate assay plates to assess reproducibility. Proliferation data was analyzed by subtracting background proliferation (PBMC+no IL-2) and means of duplicate samples calculated. Cytokine data was derived from cell culture supernatants removed from assay wells containing PBMC and pooled to obtain the mean cytokine level in the duplicate set up. TNF-α levels were quantitated at pg/ml, based on a standard curve of purified TNF-α contained in the ELISA kit. Data were further compiled for the panel of six normal human donors as outlined in the schematic shown in FIG. 1.

Cytotoxicity Assay (NK/LAK/ADCC)

In this assay, PBMC are separated from whole blood using density gradient centrifugation. PBMC are stimulated for 3 days in the presence of 10 nM IL-2 control or IL-2 mutein of interest, to generate LAK activity as generally practiced in current state of the art (see for example Isolation of Human NK Cells and Generation of LAK activity IN: Current Protocols in Immunology; 1996 John Wiley & Sons, Inc). The resulting cell population contains “effector” cells, which may be classified as NK or LAK, and can kill K562 and Daudi tumor cell targets, respectively. These effector cells may also mediate ADCC, whereby the effector cells recognize the Fc portion of a specific antibody (in this case Rituxan®) that is bound to the Daudi target cells. The assay involves co-incubation of effector cells with calcein AM-labeled target cells at various effector to target cell (E:T ratios) for 4 h. The amount of cytotoxic activity is related to the detection of calcein AM in the culture supernatant. Quantitation is expressed as percent specific lysis at each E:T ratio, based upon determination of spontaneous and maximum release controls. In summary, the assay examines the following biological activities:

| ACTIVITY | EFFECTOR | TARGET | DESCRIPTION |
|---|---|---|---|
| NK | PBMC | K562 | Natural cytotoxicty |
| LAK | PBMC | Daudi | IL-2 activated cells |
| ADCC | PBMC | Daudi + Rituxan | Antibody- |
| | | | dependent |

Data Analysis Data is obtained from the fluorimeter and expressed in relative fluorescence units (rfu). Controls include labeled target cells alone (min) and labeled target cells with final 1% Triton X-100 as a measure of 100% lysis (max). The percent min to max ratio is calculated using the following equation as a measure of assay validity (assay invalid if>30%):

$$\% min to max = 100 ⨯ \frac{mean spontaneous release rfu}{mean maximum release rfu}$$

Once the assay is deemed valid, the mean and standard deviation for triplicate sample points is calculated, followed by the percent specific lysis from mean of triplicate points using the following equation:

$$\% lysis = 100 ⨯ \frac{mean experimental rfu - mean spontaneous release rfu}{mean maximal release rfu - mean spontaneous release rfu}$$

Data is reported as % specific lysis; in addition the ratio of IL-2 mutein to relevant IL-2 control was used to determine whether cytotoxic activity was maintained relative to control IL-2 in a mixed population of human PBMC donors.
Results

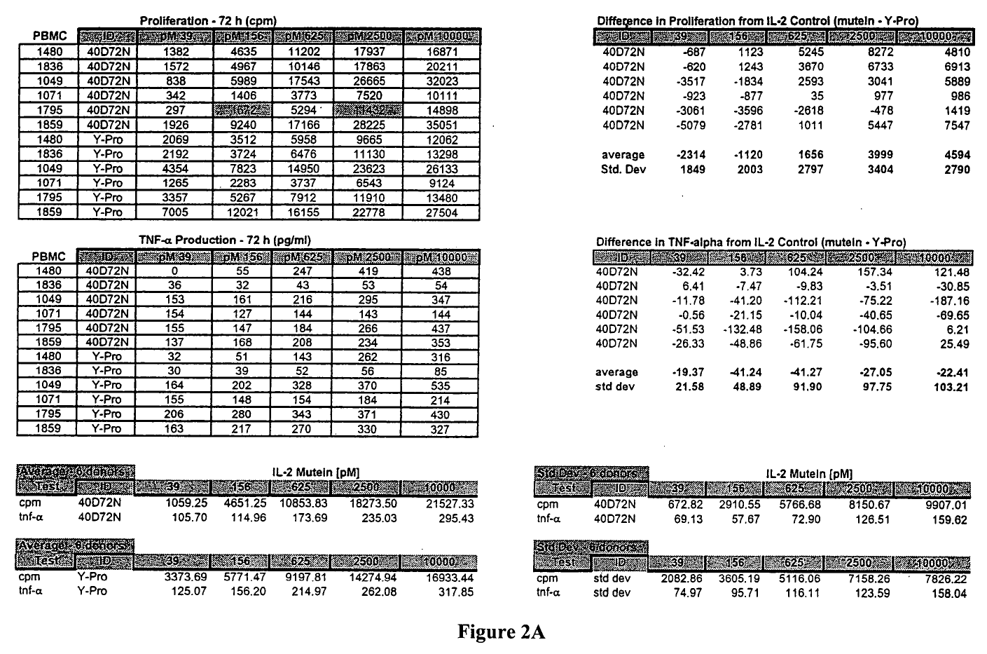

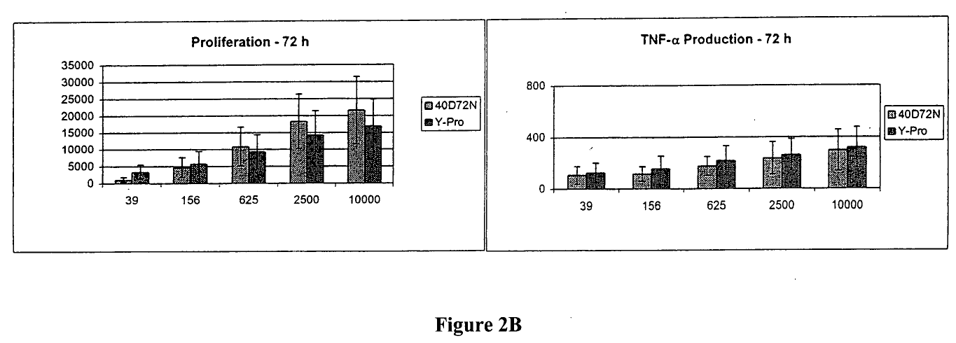

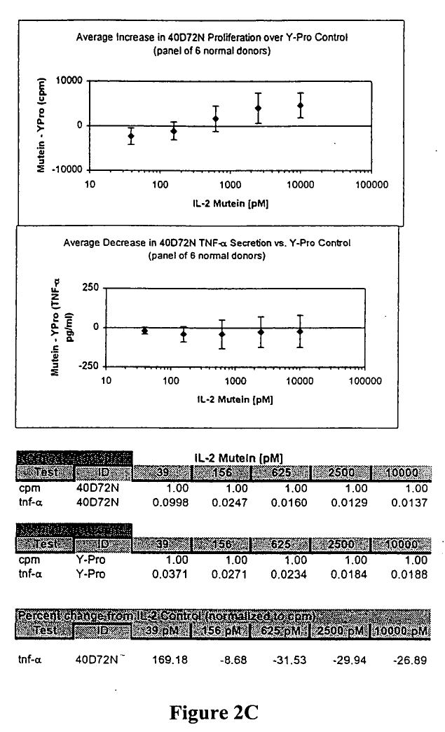

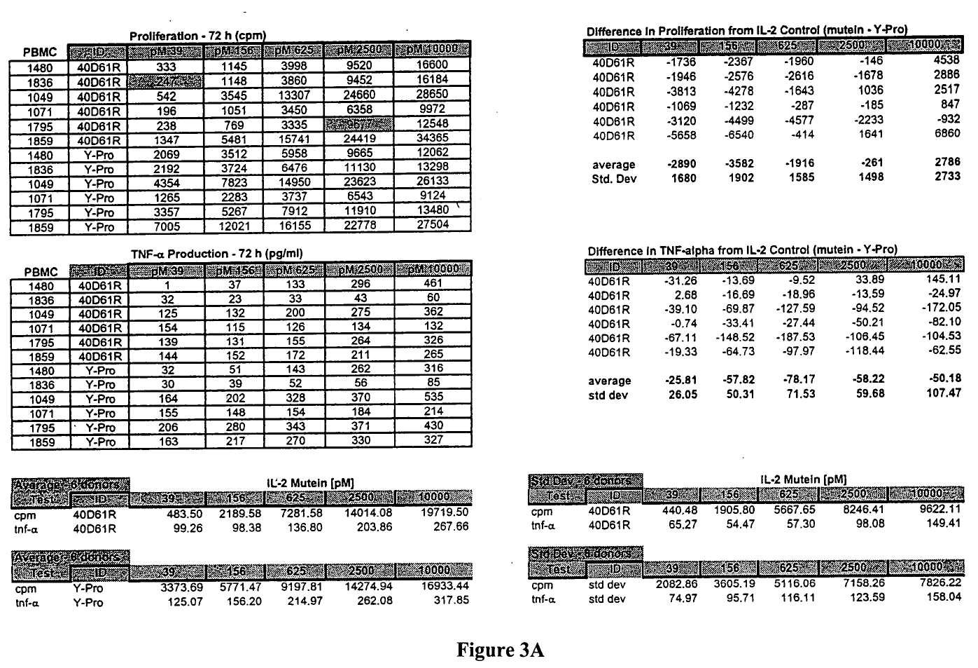

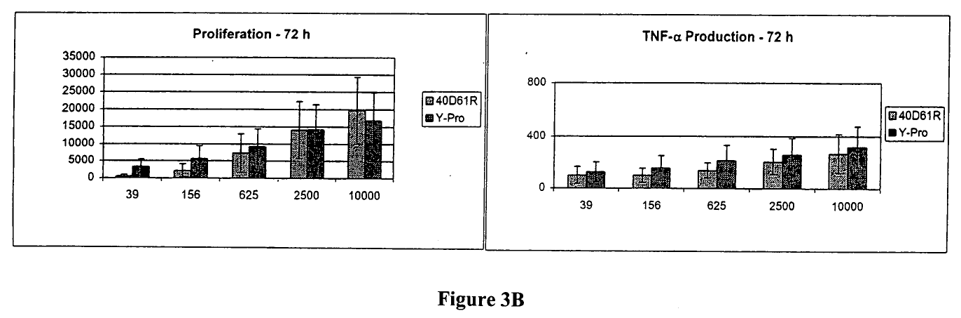

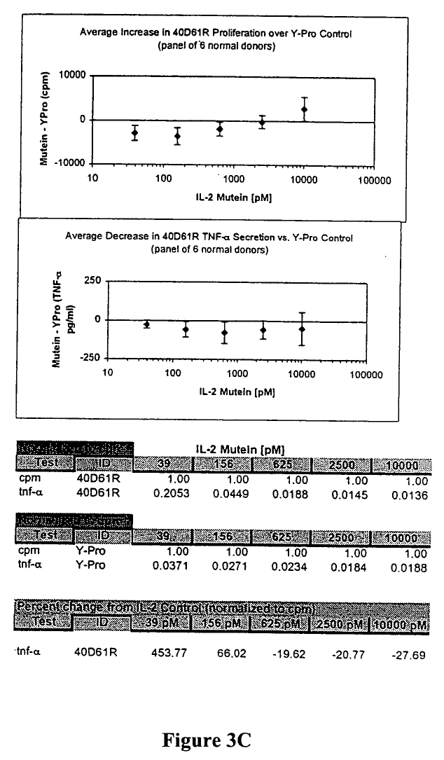

Two beneficial combinatorial IL-2 mutations that reduce pro-inflammatory cytokine production while maintaining or increasing levels of proliferation and cytotoxicity in normal human PBMC were identified: 40D72N and 40D61R. For the data set presented below, IL-2 muteins were tested along with the relevant control, i.e., des-alanyl-1, C125S human IL-2 expressed and purified in the same yeast system (designated Y-Pro). Initially IL-2 muteins were tested in the combination proliferation/pro-inflammatory cytokine production assay over a dose response curve (39 pM-10 nM) in two independent assay setups, each with three normal blood donor PBMC tested in duplicate. Data analysis included individual donor profiles, mean±standard deviation, analysis of differences from internal IL-2 controls, and normalization of cytokine production (pg/ml) to proliferation (cpm) to derive relative levels of cytokine produced per cell. Finally, the percent decrease in TNF-α production from the IL-2 control was calculated. IL-2 muteins with a decrease in TNF-α production greater than 25% at 10,000 pM were deemed beneficial if levels of proliferation were maintained. Table 9 summarizes the percent decrease in TNF-α production observed for the 2 beneficial combinatorial IL-2 muteins, which had the indicated additional combination of amino acid substitutions in the des-alanyl-1, C125S human IL-2 mutein backbone. FIGS. 2 and 3 show the proliferation and TNF-α production mediated by the 40D72N and 40D61R muteins, respectively, in human PBMC.

TABLE 9
Percent decrease in TNF- α production from IL-2 control¹.

| ID | 625 pM | 2500 pM | 10,000 pM |
|---|---|---|---|
| 40D72N | −31.53 | −29.94 | −26.89 |
| 40D61R | −19.62 | −20.77 | −27.69 |

¹Values represent average percent decrease from Y-Pro control from panel of 6 normal human PBMC donors. Cytokine data was normalized to proliferation.

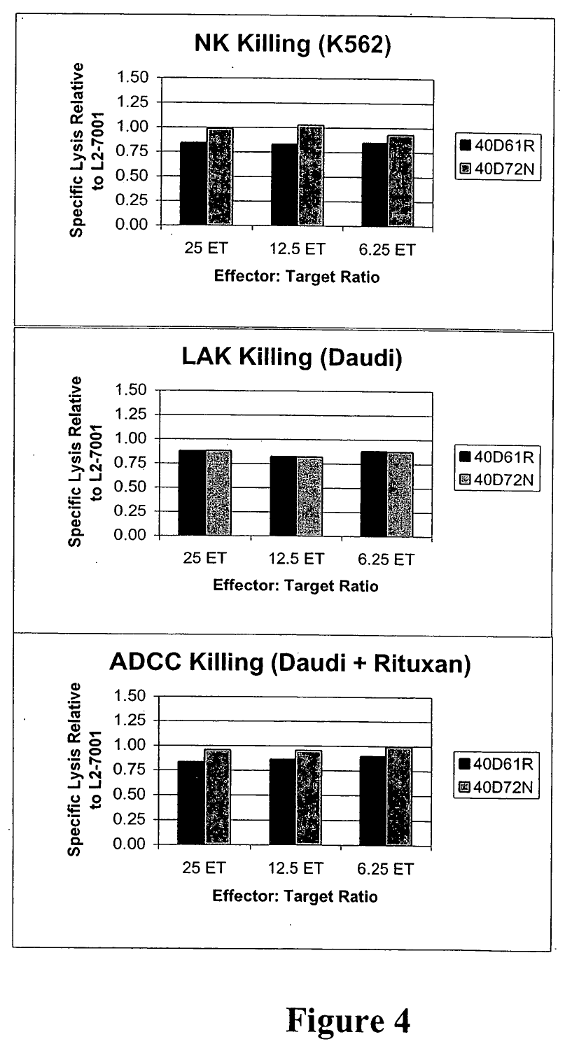

Once the 2 beneficial IL-2 muteins were identified, it was important to determine whether PBMC stimulated with IL-2 mutein retained the capacity to lyse tumor cell targets by NK, LAK, and ADCC activity. As indicated in FIG. 4, there was no difference observed between either the 40D72N IL-2 mutein or 40D61R IL-2 mutein and the relevant IL-2 control in the ability to lyse tumor targets by LAK and ADCC activity.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the embodiments disclosed herein. Although specific terms are employed herein, they are used in a generic and descriptive sense only and not for purposes of limitation.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

That which is claimed:

1. An isolated nucleic acid molecule comprising a nucleotide sequence selected from the group consisting of:
a) a nucleotide sequence encoding a mutein of human IL-2, said mutein comprising an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72;
b) the nucleotide sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71;
c) a nucleotide sequence encoding a mutein of human IL-2, said mutein comprising an amino acid sequence comprising residues 2-133 of a sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72;
d) a nucleotide sequence comprising nucleotides 4-399 of a sequence selected from the group consisting of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71;
e) a nucleotide sequence of any one of a), b), c), or d), wherein said sequence comprises a substitution of nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71 with a triplet codon that encodes alanine;
f) a nucleotide sequence of any one of a), b), c), or d), wherein said sequence comprises a substitution of nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, or 71 with a triplet codon that encodes cysteine; and
g) a nucleotide sequence of a), b), c), d), e), or f), wherein one or more codons encoding said mutein has been optimized for expression in a host cell of interest.

2. The isolated nucleic acid molecule of claim 1, wherein the nucleotide sequence of g) is selected from the group consisting of the sequence of SEQ ID NO:73, nucleotides 4-399 of SEQ ID NO:73, the sequence of SEQ ID NO:74, and nucleotides 4-399 of SEQ ID NO:74.

3. An expression vector comprising the nucleic acid molecule of claim 1.

4. A host cell comprising the nucleic acid molecule of claim 1.

5. An isolated polypeptide comprising an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
b) an amino acid sequence comprising residues 2-133 of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
c) the amino acid sequence of a) or b), wherein said sequence comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

6. An isolated polypeptide comprising a mutein of human IL-2, wherein said mutein comprises the amino acid sequence set forth in SEQ ID NO:4 with a serine substituted for cysteine at position 125 of SEQ ID NO:4 and at least two additional amino acid substitutions within SEQ ID NO:4, wherein said mutein: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2 under comparable assay conditions, wherein proliferation of said NK cells and pro-inflammatory cytokine production by said NK cells are assayed using the NK-92 bioassay.

7. The isolated polypeptide of claim 6, wherein said mutein further comprises a deletion of alanine at position 1 of SEQ ID NO:4.

8. The isolated polypeptide of claim 6, wherein said additional substitutions within SEQ ID NO:4 are selected from the group consisting of the 19D40D, 19D81K, 36D42R, 36D61R, 36D65L, 40D36D, 40D61R, 40D65Y, 40D72N, 40G36D, 40G65Y, 80K36D, 80K65Y, 81K36D, 81K42E 81K61R, 81K65Y, 81K72N, 81K88D, 81K91D, 81K107H, 81L107H, 91N95G, 107H36D, 107H42E, 107H65Y, 10R36D, 107R72N, 40D81K107H, 40G81K107H, and 91N94Y95G combination substitutions.

9. The isolated polypeptide of claim 8, wherein said mutein further comprises a deletion of alanine at position 1 of SEQ ID NO:4.

10. The isolated polypeptide of claim 6, wherein said pro-inflammatory cytokine is TNF-α.

11. The isolated polypeptide of claim 6, wherein said mutein provides maintained or improved human NK cell-mediated natural killer cytotoxicity, lymphokine activated killer (LAK) cytotoxicity, or ADCC-mediated cytotoxicity relative to that observed for a similar amount of des-alanyl-1, C125S human IL-2 mutein or C125S human IL-2 under comparable assay conditions, wherein said NK cell-mediated cytotoxicity is assayed using the NK3.3 cytotoxicity bioassay.

12. The isolated polypeptide of claim 6, wherein said NK cell proliferation induced by said mutein is greater than 150% of that induced by a similar amount of des-alanyl-1, C125S human IL-2 or C125S human IL-2 under comparable assay conditions.

13. The isolated polypeptide of claim 12, wherein said NK cell proliferation induced by said mutein is greater than 170% of that induced by des-alanyl-1, C125S human IL-2 or C125S human IL-2.

14. The isolated polypeptide of claim 13, wherein said NK cell proliferation induced by said mutein is about 200% to about 250% of that induced by des-alanyl-1, C125S human IL-2 or C125S human IL-2.

15. The isolated polypeptide of claim 6, wherein said NK cell proliferation induced by said mutein is increased by at least 10% over that induced by a similar amount of des-alanyl-1, C125S human IL-2 or C125S human IL-2 under comparable assay conditions.

16. The isolated polypeptide of claim 15, wherein said NK cell proliferation induced by said mutein is increased by at least 15% over that induced by des-alanyl-1, C125S human IL-2 or C125S human IL-2.

17. The isolated polypeptide of claim 16, wherein said pro-inflammatory cytokine production induced by said mutein is less than 100% of that induced by a similar amount of des-alanyl-1, C125S human IL-2 or C125S human IL-2 under similar assay conditions.

18. The isolated polypeptide of claim 17, wherein said pro-inflammatory cytokine production induced by said mutein is less than 70% of that induced by des-alanyl-1, C125S human IL-2 or C125S human IL-2.

19. An isolated polypeptide comprising a mutein of human IL-2, wherein said mutein comprises the amino acid sequence set forth in SEQ ID NO:4 with a serine substituted for cysteine at position 125 of SEQ ID NO:4 and at least two additional amino acid substitutions within SEQ ID NO:4, wherein the ratio of IL-2-induced NK cell proliferation to IL-2-induced TNF-α production of said mutein is at least 1.5-fold greater than that observed for a similar amount of des-alanyl-1, C125S human IL-2 mutein or C125S human IL-2 mutein under comparable assay conditions, wherein NK cell proliferation at 0.1 nM mutein and TNF-α production at 1.0 nM mutein are assayed using the NK-92 bioassay.

20. The isolated polypeptide of claim 19, wherein said ratio is at least 2.5-fold greater than that observed for des-alanyl-1, C125S human IL-2 or C125S human IL-2.

21. The isolated polypeptide of claim 19, wherein said ratio is at least 3.0-fold greater than that observed for des-alanyl-1, C125S human IL-2 or C125S human IL-2.

22. The isolated polypeptide of claim 19, wherein said mutein further comprises a deletion of alanine at position 1 of SEQ ID NO:4.

23. An isolated polypeptide comprising an amino acid sequence for a mutein of human IL-2, wherein said mutein comprises the amino acid sequence set forth in SEQ ID NO:4 with a serine substituted for cysteine at position 125 of SEQ ID NO:4 and with at least two additional amino acid substitutions, wherein said additional substitutions reside at positions of SEQ ID NO:4 selected from the group consisting of positions 19, 36, 40, 42, 61, 65, 72, 80, 81, 88, 91, 95, and 107.

24. The isolated polypeptide of claim 23, wherein said mutein further comprises a deletion of alanine at position 1 of SEQ ID NO:4.

25. The isolated polypeptide of claim 23, wherein said additional substitutions within SEQ ID NO:4 are selected from the group consisting of the 19D40D, 19D81K, 36D42R, 36D61R, 36D65L, 40D36D, 40D61R, 40D65Y, 40D72N, 40D80K, 40G36D, 40G65Y, 80K36D, 80K65Y, 81K36D, 81K42E 81K61R, 81K65Y, 81K72N, 81K88D, 81K91D, 81K107H, 81L107H, 91N95G, 107H36D, 107H42E, 107H65Y, 107R36D, 107R72N, 40D81K107H, 40G81K107H, and 91N94Y95G combination substitutions.

26. The isolated polypeptide of claim 25, wherein said mutein further comprises a deletion of alanine at position 1 of SEQ ID NO:4.

27. A method of producing a mutein of human interleukin-2 (IL-2) that is capable of maintaining or enhancing proliferation of NK cells and which also induces a lower level of pro-inflammatory cytokine production by NK cells as compared with a similar amount of a reference IL-2 mutein selected from des-alanyl-1, C125S human IL-2 and C125 human IL-2 under similar assay conditions, wherein said NK cell proliferation and pro-inflammatory cytokine production are assayed using the NK-92 bioassay, said method comprising:
a) transforming a host cell with an expression vector comprising a nucleic acid molecule of claim 1;
b) culturing said host cell in a cell culture medium under conditions that allow expression of said nucleic acid molecule as a polypeptide; and
c) isolating said polypeptide.

28. A method of producing a mutein of human interleukin-2 (IL-2) that is capable of maintaining or enhancing proliferation of NK cells and which also induces a lower level of pro-inflammatory cytokine production by NK cells as compared with a similar amount of a reference IL-2 mutein selected from des-alanyl-1, C125S human IL-2 and C125 human IL-2 under similar assay conditions, wherein said NK cell proliferation and said pro-inflammatory cytokine production are assayed using the NK-92 bioassay, said method comprising:
a) transforming a host cell with an expression vector comprising a nucleic acid molecule encoding the polypeptide of claim 23;
b) culturing said host cell in a cell culture medium under conditions that allow expression of said nucleic acid molecule as a polypeptide; and
c) isolating said polypeptide.

29. A pharmaceutical composition comprising a therapeutically effective amount of a human IL-2 mutein of claim 5 and a pharmaceutically acceptable carrier.

30. A pharmaceutical composition comprising a therapeutically effective amount of a human IL-2 mutein of claim 6 and a pharmaceutically acceptable carrier.

31. A pharmaceutical composition comprising a therapeutically effective amount of a human IL-2 mutein of claim 19 and a pharmaceutically acceptable carrier.

32. A pharmaceutical composition comprising a therapeutically effective amount of a human IL-2 mutein of claim 23 and a pharmaceutically acceptable carrier.

33. A method for stimulating the immune system of a mammal, comprising administering to said mammal a therapeutically effective amount of a human IL-2 mutein, wherein said mutein induces a lower level of pro-inflammatory cytokine production by NK cells and maintains or enhances NK cell proliferation compared to a similar amount of a reference IL-2 mutein selected from des-alanyl-1, C125S human IL-2 and C125S human IL-2 under comparable assay conditions, wherein said NK cell proliferation and said pro-inflammatory cytokine production are assayed using the NK-92 bioassay.

34. The method of claim 33, wherein said mammal is a human.

35. The method of claim 33, wherein said human IL-2 mutein comprises an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
b) an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
c) the amino acid sequence of a) or b), wherein said sequence comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

36. A method for treating a cancer in a mammal, comprising administering to said mammal a therapeutically effective amount of a human IL-2 mutein, wherein said mutein induces a lower level of pro-inflammatory cytokine production by NK cells and maintains or enhances NK cell proliferation compared to a similar amount of a reference IL-2 mutein selected from des-alanyl-1, C125S human IL-2 and C125S human IL-2 under similar assay conditions, wherein said NK cell proliferation and said pro-inflammatory cytokine production are assayed using the NK-92 bioassay.

37. The method of claim 36, wherein said mammal is a human.

38. The method of claim 36, wherein said human IL-2 mutein comprises an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
b) an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
c) the amino acid sequence of a) or b), wherein said sequence comprises an alanine residue substituted for the serine residue at position 125 of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.

39. A method for reducing interleukin-2 (IL-2)-induced toxicity symptoms in a subject undergoing IL-2 administration as a treatment protocol, said method comprising administering said IL-2 as an IL-2 mutein, wherein said IL-2 mutein comprises an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
b) an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72;
c) the amino acid sequence of a) or b), wherein said sequence comprises an alanine residue substituted for the serine residue at position 125 of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, or 72.


SEQUENCE LISTING (74 sequences)

SEQ ID NO: 1 - DNA, 459 bp, Homo sapiens
  CDS (1)...(459)
  misc_feature (0)...(0): human IL-2 precursor
atgtacaggatgcaactcctgtcttgcattgcactaagtcttgcacttgtcgcaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcagagcatcatctcaacactgact

SEQ ID NO: 2 - protein, 153 aa, Homo sapiens [sequence omitted]

SEQ ID NO: 3 - DNA, 399 bp, Homo sapiens
  CDS (1)...(399)
  misc_feature (0)...(0): mature human IL-2
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcagagcatcatctcaacactgact

SEQ ID NO: 4 - protein, 133 aa, Homo sapiens [sequence omitted]

SEQ ID NO: 5 - DNA, 399 bp, Artificial Sequence
  CDS (1)...(399): C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 6 - protein, 133 aa, Artificial Sequence
  C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - DNA, 396 bp, Artificial Sequence
  CDS (1)...(396): des-alanyl 1, C125S human IL-2 mutein
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 8 - protein, 132 aa, Artificial Sequence
  des-alanyl 1, C125S human IL-2 mutein
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - DNA, 399 bp, Artificial Sequence
  CDS (1)...(399): 19D40D, C125S human IL-2 mutein
gcacctacttcaagttctacaaagaaaacacagctacaactggagcatttactggatgatttacagatgattttgaatggaattaataattacaagaatcccaaactcaccaggatggacacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 10 - protein, 133 aa, Artificial Sequence
  19D40D, C125S human IL-2 mutein
APTSSSTKKTQLQLEHLLDDLQMILNGINNYKNPKLTRMDTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - DNA, 399 bp, Artificial Sequence
  CDS (1)...(399): 19D81K, C125S human IL-2 mutein
gcacctacttcaagttctacaaagaaaacacagctacaactggagcatttactggatgatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 12 - protein, 133 aa, Artificial Sequence
  19D81K, C125S human IL-2 mutein
APTSSSTKKTQLQLEHLLDDLQMILNGINNYKNPKLTRMLTFKFYMPKKATELKHLQCLEEELKPLEEVLNLAQSKNFHLKPRDLISNINVIVLELKGSETTFMCEYADETATIVEFLNRWITFSQSIISTLT

SEQ ID NO: 13 - DNA, 399 bp, Artificial Sequence
  CDS (1)...(399): 36D42R, C125S human IL-2 mutein
gcacctacttcaagttctacaaagaaaacacagctacaactggagcatttactgctggatttacagatgattttgaatggaattaataattacaagaatcccaaagacaccaggatgctcacaagaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 14 - protein, 133 aa, Artificial Sequence
  36D42R, C125S human IL-2 mutein
APTSSSTKKTQLQLEHLLLDLQMILNGINNYKNPKDTRMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 15 - DNA, 399 bp, Artificial Sequence
  CDS (1)...(399): 36D61R, C125S human IL-2 mutein
gcacctacttcaagttctacaaagaaaacacagctacaactggagcatttactgctggatttacagatgattttgaatggaattaataattacaagaatcccaaagacaccaggatgctcacatttaagttttacatgcccaagaaggccacagaactgaaacatcttcagtgtctagaaagagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 16 - protein, 133 aa, Artificial Sequence
  36D61R, C125S human IL-2 mutein
APTSSSTKKTQLQLEHLLLDLQMILNGINNYKNPKDTRMLTFKFYMPKKATELKHLQCLERELKPLEEVLNLAQSKNFHLRPRDLISNINVIVLELKGSETTFMCEYADETATIVEFLNRWITFSQSIISTLT

SEQ ID NO: 17 - DNA, 399 bp, Artificial Sequence
  CDS (1)...(399): 36D65L, C125S human IL-2 mutein
gcacctacttcaagttctacaaagaaaacacagctacaactggagcatttactgctggatttacagatgattttgaatggaattaataattacaagaatcccaaagacaccaggatgctcacatttaagttttacatgcccaagaaggccacagaactgaaacatcttcagtgtctagaagaagaactcaaattgctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 18 - protein, 133 aa, Artificial Sequence
  36D65L, C125S human IL-2 mutein
APTSSSTKKTQLQLEHLLLDLQMILNGINNYKNPKDTRMLTFKFYMPKKATELKHLQCLEEELKLLEEVLNLAQSKNFHLRPRDLISNINVIVLELKGSETTFMCEYADETATIVEFLNRWITFSQSIISTLT

SEQ ID NO: 19 - DNA, 399 bp, Artificial Sequence
  CDS (1)...(399): 40D36D, C125S human IL-2 mutein
gcacctacttcaagttctacaaagaaaacacagctacaactggagcatttactgctggatttacagatgattttgaatggaattaataattacaagaatcccaaagacaccaggatggacacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 20 - protein, 133 aa, Artificial Sequence
  40D36D, C125S human IL-2 mutein
APTSSSTKKTQLQLEHLLLDLQMILNGINNYKNPKDTRMDTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 21 - DNA, 399 bp, Artificial Sequence
  CDS (1)...(399): 40D61R, C125S human IL-2 mutein
gcacctacttcaagttctacaaagaaaacacagctacaactggagcatttactgctggatttacagatgattttgaatggaattaataattacaagaatcccaaactcaccaggatggacacatttaagttttacatgcccaagaaggccacagaactgaaacatcttcagtgtctagaaagagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 22 - protein, 133 aa, Artificial Sequence
  40D61R, C125S human IL-2 mutein
APTSSSTKKTQLQLEHLLLDLQMILNGINNYKNPKLTRMDTFKFYMPKKATELKHLQCLERELKPLEEVLNLAQSKNFHLRPRDLISNINVIVLELKGSETTFMCEYADETATIVEFLNRWITFSQSIISTLT

SEQ ID NO: 23 - DNA, 399 bp, Artificial Sequence
  CDS (1)...(399): 40D65Y, C125S human IL-2 mutein
gcacctacttcaagttctacaaagaaaacacagctacaactggagcatttactgctggatttacagatgattttgaatggaattaataattacaagaatcccaaactcaccaggatggacacatttaagttttacatgcccaagaaggccacagaactgaaacatcttcagtgtctagaagaagaactcaaatacctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 24 - protein, 133 aa, Artificial Sequence
  40D65Y, C125S human IL-2 mutein
APTSSSTKKTQLQLEHLLLDLQMILNGINNYKNPKLTRMDTFKFYMPKKATELKHLQCLEEELKYLEEVLNLAQSKNFHLRPRDLISNINVIVLELKGSETTFMCEYADETATIVEFLNRWITFSQSIISTLT

SEQ ID NO: 25 - DNA, 399 bp, Artificial Sequence
  CDS (1)...(399): 40D72N, C125S human IL-2 mutein
gcacctacttcaagttctacaaagaaaacacagctacaactggagcatttactgctggatttacagatgattttgaatggaattaataattacaagaatcccaaactcaccaggatggacacatttaagttttacatgcccaagaaggccacagaactgaaacatcttcagtgtctagaagaagaactcaaacctctggaggaagtgctaaataacgctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 26 - protein, 133 aa, Artificial Sequence
  40D72N, C125S human IL-2 mutein
APTSSSTKKTQLQLEHLLLDLQMILNGINNYKNPKLTRMDTFKFYMPKKATELKHLQCLEEELKPLEEVLNNAQSKNFHLRPRDLISNINVIVLELKGSETTFMCEYADETATIVEFLNRWITFSQSIISTLT

SEQ ID NO: 27 - DNA, 399 bp, Artificial Sequence
  CDS (1)...(399): 40D80K, C125S human IL-2 mutein
gcacctacttcaagttctacaaagaaaacacagctacaactggagcatttactgctggatttacagatgattttgaatggaattaataattacaagaatcccaaactcaccaggatggacacatttaagttttacatgcccaagaaggccacagaactgaaacatcttcagtgtctagaagaagaactcaaacctctggaggaagtgctaaatttagctcaaagcaaaaactttcacaagagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 28 - protein, 133 aa, Artificial Sequence
  40D80K, C125S human IL-2 mutein
APTSSSTKKTQLQLEHLLLDLQMILNGINNYKNPKLTRMDTFKFYMPKKATELKHLQCLEEELKPLEEVLNLAQSKNFHKRPRDLISNINVIVLELKGSETTFMCEYADETATIVEFLNRWITFSQSIISTLT

SEQ ID NO: 29 - DNA, 399 bp, Artificial Sequence
  CDS (1)...(399): 40G36D, C125S human IL-2 mutein
gcacctacttcaagttctacaaagaaaacacagctacaactggagcatttactgctggatttacagatgattttgaatggaattaataattacaagaatcccaaagacaccaggatgggtacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 30 - protein, 133 aa, Artificial Sequence
  40G36D, C125S human IL-2 mutein
APTSSSTKKTQLQLEHLLLDLQMILNGINNYKNPKDTRMGTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 31 - DNA, 399 bp, Artificial Sequence
  CDS (1)...(399): 40G65Y, C125S human IL-2 mutein
gcacctacttcaagttctacaaagaaaacacagctacaactggagcatttactgctggatttacagatgattttgaatggaattaataattacaagaatcccaaactcaccaggatgggtacatttaagttttacatgcccaagaaggccacagaactgaaacatcttcagtgtctagaagaagaactcaaatacctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 32 - protein, 133 aa, Artificial Sequence
  40G65Y, C125S human IL-2 mutein
APTSSSTKKTQLQLEHLLLDLQMILNGINNYKNPKLTRMGTFKFYMPKKATELKHLQCLEEELKYLEEVLNLAQSKNFHLRPRDLISNINVIVLELKGSETTFMCEYADETATIVEFLNRWITFSQSIISTLT

SEQ ID NO: 33 - DNA, 399 bp, Artificial Sequence
  CDS (1)...(399): 80K36D, C125S human IL-2 mutein
gcacctacttcaagttctacaaagaaaacacagctacaactggagcatttactgctggatttacagatgattttgaatggaattaataattacaagaatcccaaagacaccaggatgctcacatttaagttttacatgcccaagaaggccacagaactgaaacatcttcagtgtctagaagaagaactcaaacctctggaggaagtgctaaatttagctcaaagcaaaaactttcacaagagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 34 - protein, 133 aa, Artificial Sequence
  80K36D, C125S human IL-2 mutein
APTSSSTKKTQLQLEHLLLDLQMILNGINNYKNPKDTRMLTFKFYMPKKATELKHLQCLEEELKPLEEVLNLAQSKNFHKRPRDLISNINVIVLELKGSETTFMCEYADETATIVEFLNRWITFSQSIISTLT

SEQ ID NO: 35 - DNA, 399 bp, Artificial Sequence
  CDS (1)...(399): 80K65Y, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaatacctggaggaagtgctaaatttagctcaaagcaaaaactttcacaagagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 36 - protein, 133 aa, Artificial Sequence
  80K65Y, C125S human IL-2 mutein
APTSSSTKKTQLQLEHLLLDLQMILNGINNYKNPKLTRMLTFKFYMPKKATELKHLQCLEEELKYLEEVLNLAQSKNFHKRPRDLISNINVIVLELKGSETTFMCEYADETATIVEFLNRWITFSQSIISTLT

SEQ ID NO: 37 - DNA, 399 bp, Artificial Sequence
  CDS (1)...(399): 81K36D, C125S human IL-2 mutein
gcacctacttcaagttctacaaagaaaacacagctacaactggagcatttactgctggatttacagatgattttgaatggaattaataattacaagaatcccaaagacaccaggatgctcacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 38 - protein, 133 aa, Artificial Sequence
  81K36D, C125S human IL-2 mutein
APTSSSTKKTQLQLEHLLLDLQMILNGINNYKNPKDTRMLTFKFYMPKKATELKHLQCLEEELKPLEEVLNLAQSKNFHLKPRDLISNINVIVLELKGSETTFMCEYADETATIVEFLNRWITFSQSIISTLT

SEQ ID NO: 39 - DNA, 399 bp, Artificial Sequence
  CDS (1)...(399): 81K42E, C125S human IL-2 mutein
gcacctacttcaagttctacaaagaaaacacagctacaactggagcatttactgctggatttacagatgattttgaatggaattaataattacaagaatcccaaactcaccaggatgctcacagaaaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 40 - protein, 133 aa, Artificial Sequence
  81K42E, C125S human IL-2 mutein
APTSSSTKKTQLQLEHLLLDLQMILNGINNYKNPKLTRMLTEKFYMPKKATELKHLQCLEEELKPLEEVLNLAQSKNFHLKPRDLISNINVIVLELKGSETTFMCEYADETATIVEFLNRWITFSQSIISTLT

SEQ ID NO: 41 - DNA, 399 bp, Artificial Sequence
  CDS (1)...(399): 81K61R, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaaagagaactcaaacctctggaggaagtgctaaatttagctcaaagcaaaaactttcacttaaagcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 42 - protein, 133 aa, Artificial Sequence
  81K61R, C125S human IL-2 mutein
APTSSSTKKTQLQLEHLLLDLQMILNGINNYKNPKLTRMLTFKFYMPKKATELKHLQCLERELKPLEEVLNLAQSKNFHLKPRDLISNINVIVLELKGSETTFMCEYADETATIVEFLNRWITFSQSIISTLT

SEQ ID NO: 43 - DNA, 399 bp, Artificial Sequence
  CDS (1)...(399): 81K65Y, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaatacctggaggaagtgctaaatttagctcaaagcaaaaactttcacttaaagcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 44 - protein, 133 aa, Artificial Sequence
  81K65Y, C125S human IL-2 mutein
APTSSSTKKTQLQLEHLLLDLQMILNGINNYKNPKLTRMLTFKFYMPKKATELKHLQCLEEELKYLEEVLNLAQSKNFHLKPRDLISNINVIVLELKGSETTFMCEYADETATIVEFLNRWITFSQSIISTLT

SEQ ID NO: 45 - DNA, 399 bp, Artificial Sequence
  CDS (1)...(399): 81K72N, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaataacgctcaaagcaaaaactttcacttaaagcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 46 - protein, 133 aa, Artificial Sequence
  81K72N, C125S human IL-2 mutein
APTSSSTKKTQLQLEHLLLDLQMILNGINNYKNPKLTRMLTFKFYMPKKATELKHLQCLEEELKPLEEVLNNAQSKNFHLKPRDLISNINVIVLELKGSETTFMCEYADETATIVEFLNRWITFSQSIISTLT

SEQ ID NO: 47 - DNA, 399 bp, Artificial Sequence
  CDS (1)...(399): 81K88D, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcgatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 48 - protein, 133 aa, Artificial Sequence
  81K88D, C125S human IL-2 mutein
APTSSSTKKTQLQLEHLLLDLQMILNGINNYKNPKLTRMLTFKFYMPKKATELKHLQCLEEELKPLEEVLNLAQSKNFHLKPRDLISDINVIVLELKGSETTFMCEYADETATIVEFLNRWITFSQSIISTLT

SEQ ID NO: 49 - DNA, 399 bp, Artificial Sequence
  CDS (1)...(399): 81K91D, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgatatagttctggaactaaagggatctgaaacaacattcatgtgtgaatatgctgatgagacagcaaccattgtagaatttctgaacagatggattaccttttctcagagcatcatctcaacactgact

SEQ ID NO: 50 - protein, 133 aa, Artificial Sequence
  81K91D, C125S human IL-2 mutein
APTSSSTKKTQLQLEHLLLDLQMILNGINNYKNPKLTRMLTFKFYMPKKATELKHLQCLEEELKPLEEVLNLAQSKNFHLKPRDLISNINDIVLELKGSETTFMCEYADETATIVEFLNRWITFSQSIISTLT

SEQ ID NO: 51 - DNA, 399 bp, Artificial Sequence
  CDS (1)...(399): 81K107H, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 52 - protein, 133 aa, Artificial Sequence
  81K107H, C125S human IL-2 mutein
APTSSSTKKTQLQLEHLLLDLQMILNGINNYKNPKLTRMLTFKFYMPKKATELKHLQCLEEELKPLEEVLNLAQSKNFHLKPRDLISNINVIVLELKGSETTFMCEHADETATIVEFLNRWITFSQSIISTLT

SEQ ID NO: 53 - DNA, 399 bp, Artificial Sequence
  CDS (1)...(399): 81L107H, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttattgcccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 54 - protein, 133 aa, Artificial Sequence
  81L107H, C125S human IL-2 mutein
APTSSSTKKTQLQLEHLLLDLQMILNGINNYKNPKLTRMLTFKFYMPKKATELKHLQCLEEELKPLEEVLNLAQSKNFHLLPRDLISNINVIVLELKGSETTFMCEHADETATIVEFLNRWITFSQSIISTLT

SEQ ID NO: 55 - DNA, 399 bp, Artificial Sequence
  CDS (1)...(399): 91N95G, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacaacatagttctgggtctaaagggatctgaaacaacattcatgtgtgaatatgctgatgagacagcaaccattgtagaatttctgaacagatggattaccttttctcagagcatcatctcaacactgact

SEQ ID NO: 56 - protein, 133 aa, Artificial Sequence
  91N95G, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINNIVLGLKGSETTFMCEYADETATIVEFLNRWITFSQSIISTLT

SEQ ID NO: 57 - DNA, 399 bp, Artificial Sequence
  CDS (1)...(399): 107H36D, C125S human IL-2 mutein
gcacctacttcaagttctacaaagaaaacacagctacaactggagcatttactgctggatttacagatgattttgaatggaattaataattacaagaatcccaaagacaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 58 - protein, 133 aa, Artificial Sequence
  107H36D, C125S human IL-2 mutein
APTSSSTKKTQLQLEHLLLDLQMILNGINNYKNPKDTRMLTFKFYMPKKATELKHLQCLEEELKPLEEVLNLAQSKNFHLRPRDLISNINVIVLELKGSETTFMCEHADETATIVEFLNRWITFSQSIISTLT

SEQ ID NO: 59 - DNA, 399 bp, Artificial Sequence
  CDS (1)...(399): 107H42E, C125S human IL-2 mutein
gcacctacttcaagttctacaaagaaaacacagctacaactggagcatttactgctggatttacagatgattttgaatggaattaataattacaagaatcccaaactcaccaggatgctcacagaaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 60 - protein, 133 aa, Artificial Sequence
  107H42E, C125S human IL-2 mutein
APTSSSTKKTQLQLEHLLLDLQMILNGINNYKNPKLTRMLTEKFYMPKKATELKHLQCLEEELKPLEEVLNLAQSKNFHLRPRDLISNINVIVLELKGSETTFMCEHADETATIVEFLNRWITFSQSIISTLT

SEQ ID NO: 61 - DNA, 399 bp, Artificial Sequence
  CDS (1)...(399): 107H65Y, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaatacctggaggaagtgctaaatttagctcaaagcaaaaactttcacttaagacccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 62 - protein, 133 aa, Artificial Sequence
  107H65Y, C125S human IL-2 mutein
APTSSSTKKTQLQLEHLLLDLQMILNGINNYKNPKLTRMLTFKFYMPKKATELKHLQCLEEELKYLEEVLNLAQSKNFHLRPRDLISNINVIVLELKGSETTFMCEHADETATIVEFLNRWITFSQSIISTLT

SEQ ID NO: 63 - DNA, 399 bp, Artificial Sequence
  CDS (1)...(399): 107R36D, C125S human IL-2 mutein
gcacctacttcaagttctacaaagaaaacacagctacaactggagcatttactgctggatttacagatgattttgaatggaattaataattacaagaatcccaaagacaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaaagagctgatgagacagcaaccattgtagaatttctgaacagatggattaccttttctcagagcatcatctcaacactgact

SEQ ID NO: 64 - protein, 133 aa, Artificial Sequence
  107R36D, C125S human IL-2 mutein
APTSSSTKKTQLQLEHLLLDLQMILNGINNYKNPKDTRMLTFKFYMPKKATELKHLQCLEEELKPLEEVLNLAQSKNFHLRPRDLISNINVIVLELKGSETTFMCERADETATIVEFLNRWITFSQSIISTLT

SEQ ID NO: 65 - DNA, 399 bp, Artificial Sequence
  CDS (1)...(399): 107R72N, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaataacgctcaaagcaaaaactttcacttaagacccagggacttaatcagcaatatcaacgtaatagttctggaactaaagggatctgaaacaacattcatgtgtgaaagagctgatgagacagcaaccattgtagaatttctgaacagatggattaccttttctcagagcatcatctcaacactgact

SEQ ID NO: 66 - protein, 133 aa, Artificial Sequence
  107R72N, C125S human IL-2 mutein
APTSSSTKKTQLQLEHLLLDLQMILNGINNYKNPKLTRMLTFKFYMPKKATELKHLQCLEEELKPLEEVLNNAQSKNFHLRPRDLISNINVIVLELKGSETTFMCERADETATIVEFLNRWITFSQSIISTLT

SEQ ID NO: 67 - DNA, 399 bp, Artificial Sequence
  CDS (1)...(399): 40D81K107H, C125S human IL-2  mutein
gcacctacttcaagttctacaaagaaaacacagctacaactggagcatttactgctggatttacagatgattttgaatggaattaataattacaagaatcccaaactcaccaggatggacacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 68 - protein, 133 aa, Artificial Sequence
  40D81K107H, C125S human IL-2  mutein
APTSSSTKKTQLQLEHLLLDLQMILNGINNYKNPKLTRMDTFKFYMPKKATELKHLQCLEEELKPLEEVLNLAQSKNFHLKPRDLISNINVIVLELKGSETTFMCEHADETATIVEFLNRWITFSQSIISTLT

SEQ ID NO: 69 - DNA, 399 bp, Artificial Sequence
  CDS (1)...(399): 40G81K107H, C125S human IL-2 mutein
gcacctacttcaagttctacaaagaaaacacagctacaactggagcatttactgctggatttacagatgattttgaatggaattaataattacaagaatcccaaactcaccaggatgggtacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 70 - protein, 133 aa, Artificial Sequence
  40G81K107H, C125S human IL-2 mutein
APTSSSTKKTQLQLEHLLLDLQMILNGINNYKNPKLTRMGTFKFYMPKKATELKHLQCLEEELKPLEEVLNLAQSKNFHLKPRDLISNINVIVLELKGSETTFMCEHADETATIVEFLNRWITFSQSIISTLT

SEQ ID NO: 71 - DNA, 399 bp, Artificial Sequence
  CDS (1)...(399): 91N94Y95G, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacaacatagtttacggtctaaagggatctgaaacaacattcatgtgtgaatatgctgatgagacagcaaccattgtagaatttctgaacagatggattaccttttctcagagcatcatctcaacactgact

SEQ ID NO: 72 - protein, 133 aa, Artificial Sequence
  91N94Y95G, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINNIVYGLKGSETTFMCEYADETATIVEFLNRWITFSQSIISTLT

SEQ ID NO: 73 - DNA, 399 bp, Artificial Sequence
  CDS (1)...(399): 36D61R, C125S human IL-2 mutein with 36D and 61R codons optimized for E. coli expression
gcacctacttcaagttctacaaagaaaacacagctacaactggagcatttactgctggatttacagatgattttgaatggaattaataattacaagaatcccaaagataccaggatgctcacatttaagttttacatgcccaagaaggccacagaactgaaacatcttcagtgtctagaacgtgaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 74 - DNA, 399 bp, Artificial Sequence
  CDS (1)...(399): 107R36D, C125S human IL-2 mutein with 107R and 36D codons optimized for E. coli expression
gcacctacttcaagttctacaaagaaaacacagctacaactggagcatttactgctggatttacagatgattttgaatggaattaataattacaagaatcccaaagataccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaacgtgctgatgagacagcaaccattgtagaatttctgaacagatggattaccttttctcagagcatcatctcaacactgact